(19) United States
(12) Patent Application Publication
(10) Pub. No.: US 2004/0132798 A1
(43) Pub. Date: Jul. 8, 2004

(54) 3.7-Diazybicyclo [3.3.1] formulations as antiarrhythmic compounds

(76) Inventors: Hans Carlsson, Molndal (SE); Anette Larsson, Molndal (SE)

Correspondence Address: Patricia Granahan, Ropes & Gray, One International Place, Boston, MA, 02110-2624, US

(21) Appl. No.: 10/474,591

(22) PCT Filed: Apr. 12, 2002
(86) PCT No.: PCT/SE02/00724

(30) Foreign Application Priority Data
Apr. 12, 2001 (SE) 0101325-9
Apr. 12, 2001 (SE) 0101326-7

(51) Int. Cl. A61K 31/407

(52) U.S. Cl. 514/412

(57) ABSTRACT

There is provided a modified release pharmaceutical composition comprising 4-({3-[7-(3,3-dimethyl-2-oxobutyl)-9-oxa-3,7-diazabicyclo-[3.3.1]non-3-yl]propyl}amino)benzonitrile, tert-butyl 2-{7-[3-(4-cyano-anilino)propyl ]-9-oxa-3,7-diazabicy-clo[3.3.1]non-3-yl}ethylcarbamate, tert-butyl 2- {7-[4-(4-cyanophenyl)butyl]-9-oxa-3,7-diazabicyclo[3.3.1]non-3-yl}ethylcarbamate, or tert-butyl 2-{7-[(2S)-3-(4-cyanophenoxy)-2-hydroxypropyl]-9-oxa-3,7-diazabicyclo[3.3.1]non-3-yl}ethylcarbamate, or a pharmaceutically-acceptable salt of any of these compounds, in particular a gelling polymer matrix modified release composition. The compositions are useful in the prophylaxis and/or treatment of cardiac arrhythmias.

### FIELD OF THE INVENTION

This invention relates to novel pharmaceutical formulations that provide for modified delivery of particular drugs, which drugs are useful in the treatment of cardiac arrhythmias.

### BACKGROUND AND PRIOR ART

It is often necessary to administer pharmaceutically-active compounds frequently throughout the day in order to maintain a desired therapeutic level of active principle in plasma, body tissues and/or the gastrointestinal tract. This is particularly the case where it is intended to deliver the drug orally and to provide a uniform response over an extended period of time.

Over the last thirty or so years, modified release dosage forms have increasingly become a preferred method of delivering certain drugs to patients, particularly via the oral route. Such forms may e.g. provide for release of drug over an extended period of time, thus reducing the number of required daily doses, and during which time the rate of release may be substantially uniform and/or constant, within a specific part of the gastrointestinal tract, or pulsative.

There are numerous modified release dosage forms known in the art and these have been summarised by inter alia De Haan and Lerk in Pharmaceutisch Weekblad Scientific Edition, 6, 57 (1984); Banker in “Medical Applications of Controlled Release”, Vol II, eds. Langer and Wise (1984) Bocaraton, Florida, at pages 1 to 34; Graffner in Industrial Aspects of Pharmaceuticals, ed. Sandel, Swedish Pharmaceutical Press (1993) at pages 93 to 104; and Proudfoot “Dosage Regimens: Their Influence on the Concentration-Time Profile of the Drug in the Body” at pages 191 to 211 of “Pharmaceutics: The Science of Dosage Form Design”, ed. M. E. Aulton (1988) (Churchill Livingstone).

International patent application WO 01/28992 discloses a series of oxabispidine compounds, including:

(a) 4-( {3-[7-(3,3-dimethyl-2-oxobutyl)-9-oxa-3,7-diazabicyclo[3.3.1]non-3-yl]propyl}amino)benzonitrile:

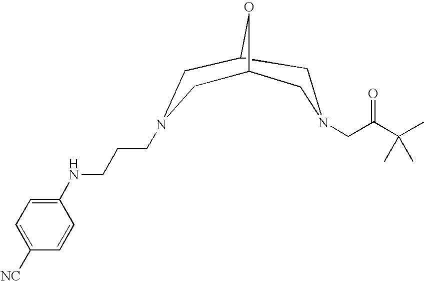

which compound is referred to hereinafter as Compound A. Compound A is specifically disclosed in WO 01/28992 both in the form of the free base and in the form of a benzenesulphonate salt;

(b) tert-butyl 2-{7-[3-(4-cyanoanilino)propyl]-9-oxa-3,7-diazabicyclo-[3.3.1]non-3-yl}ethylcarbamate:

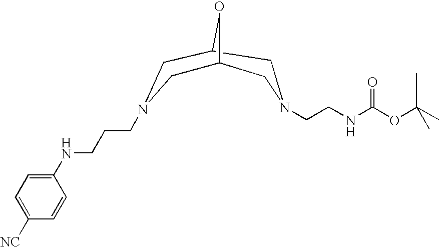

in the form of the free base, which compound is referred to hereinafter as Compound B;

(c) tert-butyl 2- {7-[4-(4-cyanophenyl)butyl]-9-oxa-3,7-diazabicyclo-[3.3.1]non-3-yl}ethylcarbamate:

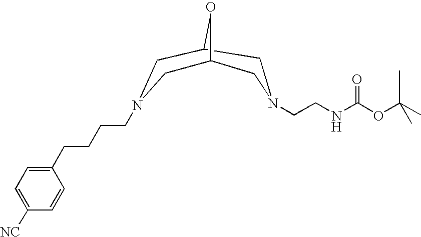

in the form of the free base, which compound is referred to hereinafter as Compound C; and

(d) tert-butyl 2- {7-[(2S)-3-(4-cyanophenoxy)-2-hydroxypropyl]-9-oxa-3,7-diazabicyclo[3.3.1]non-3-yl}ethylcarbarmate:

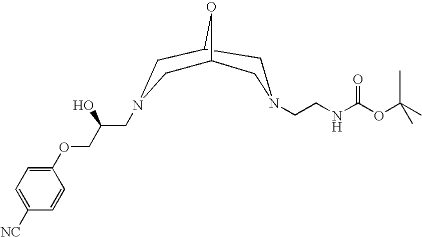

in the form of the free base, which compound is referred to hereinafter as Compound D.

The compounds of international patent application WO 01/28992 are indicated as being useful in the treatment of cardiac arrhythmias.

Although general information is provided in WO 01/28992 in relation to how the compounds disclosed therein may be formulated and thereafter administered to patients, no mention is made of modified release pharmaceutical formulations including, specifically, Compounds A, B, C or D and salts thereof.

We have found that it may be advantageous to provide Compounds A, B, C and D, and pharmaceutically-acceptable salts of any of these compounds, in a modified release dosage form.

### DESCRIPTION OF THE INVENTION

According to the invention there is provided a modified release pharmaceutical composition (formulation) comprising, as active ingredient, Compound A, Compound B, Compound C or Compound D, or a pharmaceutically-acceptable salt of any of Compounds A, B, C or D, which compositions are referred to hereinafter as “the compositions of the invention”.

Compounds A, B, C and D, as well as pharmaceutically-acceptable salts of these compounds, may be prepared as described in WO 01/28992, as described hereinafter and/or by way of routine techniques in organic chemistry. Compositions comprising solvates, including hydrates, as well as anhydrates (and ansolvates) of Compounds A, B, C, D, and pharmaceutically-acceptable salts of these compounds, are also included within the scope of the invention.

The term “modified release” pharmaceutical composition will be well understood by the skilled person to include any composition/formulation in which the onset and/or rate of release of drug (whether in the form of Compound A, Compound B, Compound C, Compound D, or as a pharmaceutically-acceptable salt of any of these compounds) is altered by galenic manipulations, and thus includes the definition provided in the United States Pharmacopeia (USP XXII) at pages xliii and xliv of the preface/preamble part, the relevant disclosure in which document is hereby incorporated by reference.

In the present case, modified release may be provided for by way of an appropriate pharmaceutically-acceptable carrier, and/or other means, which carrier or means (as appropriate) gives rise to an alteration of the onset and/or rate of release of active ingredient. Thus, the term will be understood by those skilled in the art to include compositions which are adapted (for example as described herein) to provide for a “sustained”, a “prolonged” or an “extended” release of drug (in which drug is released at a sufficiently retarded rate to produce a therapeutic response over a required period of time, optionally including provision for an initial amount of drug being made available within a predetermined time following administration to cause an initial desired therapeutic response); compositions which provide for a “delayed” release of drug (in which the release of drug is delayed until a specific region of the gastrointestinal tract is reached, following which drug release may be either pulsatile or further modified as indicated above); as well as so-called “repeat action” compositions (in which one dose of drug is released either immediately or some time after administration and further doses are released at a later time).

We prefer that the compositions of the invention provide for a delayed release or, more preferably, a sustained (i.e. prolonged or extended) release of drug over a period of time. More preferred compositions of the invention may be is adapted (for example as described herein) to provide a sufficient dose of drug over the dosing interval (irrespective of the number of doses per unit time) to produce a desired therapeutic effect. Release may be uniform and/or constant over an extended period of time, or otherwise.

Compositions of the invention may, for example, be in the form of the following, all of which are well known to those skilled in the art:

(a) Coated pellets, tablets or capsules, which may be designed to release at least some of the drug when the formulation in question reaches a particular region of the gastrointestinal tract. Such tablets may, for example be provided with some form of gastro-resistant coating, such as an enteric coating layer, providing for release of at least part of the drug present in the formulation in a specific part of the gastrointestinal tract, such as the intestinal regions.

(b) Multiple unit or multiparticulate systems, which may be in the form of microparticles, microspheres or pellets comprising drug (which multiple units/multiparticulates may provide for gradual emptying of the formulation containing drug from the stomach into the duodenum and further through the small and large intestine while releasing drug at a pre-determined rate).

(c) Formulations comprising dispersions or solid solutions of active compound in a matrix, which may be in the form of a wax, gum or fat, or, particularly, in the form of a polymer, in which drug release takes place by way of gradual surface erosion of the tablet and/or diffusion.

(d) Systems which comprise a bioadhesive layer, which layer may provide for prolonged retention of composition of the invention in a particular region of the gastrointestinal tract (e.g. the stomach). This includes floating or sinking systems (i.e. low and high density systems, respectively), as well as so-called “volume-enlarging” systems.

(e) So-called, “pendent” devices, in which drug is attached to an ion exchange resin, which provides for gradual release of drug by way of influence of other ions present in the gastrointestinal tract, for example, the acid environment of the stomach.

(f) Devices in which release rate of drug is controlled by way of its chemical potential (e.g. the Osmotic Pump).

(g) Systems in which drug is released by diffusion through membranes, including multilayer systems.

(h) Devices that act in accordance with an external signal, to release a small amount of drug.

(i) Active, self-programmed systems, which may contain a sensing element, which element responds to a particular biological environment to modulate drug delivery.

(j) Silastic controlled release depots, which release drug as a finction of diffusion of water and/or gastrointestinal fluids into the device via an entry/exit port, resulting in dissolution and subsequent release of drug.

(k) Combinations of two or more of the above principles.

The above principles are discussed at length in numerous prior art references including Pharmaceutisch Weekblad Scientific Edition, 6, 57 (1984); Medical Applications of Controlled Release, Vol II, eds. Langer and Wise (1984) Bocaraton, Florida, at pages 1 to 34; Industrial Aspects of Pharmaceuticals, ed. Sandel, Swedish Pharmaceutical Press (1993) at pages 93 to 104; and pages 191 to 211 of “Pharmaceutics: The Science of Dosage Form Design”, ed. M. E. Aulton (1988) (Churchill Livingstone); as well as the references cited in the above-mentioned documents, the disclosures in all of which documents are hereby incorporated by reference.

Suitable modified release formulations may thus be prepared by the skilled person in accordance with standard techniques in pharmacy, as described herein or in the above-mentioned documents, and/or which are well known.

We prefer that, in the compositions of the invention, active ingredient is provided together with a pharmaceutically-acceptable carrier. In particular, we prefer that compositions of the invention are presented in the form of active ingredient embedded in a polymer matrix.

In this respect, we prefer that the compositions of the invention are provided for oral administration in the form of a so-called “swelling” modified-release system, or a “gelling matrix” modified-release system, in which active ingredient is provided together with a polymer that swells in an aqueous medium (i.e. a “hydrophilic gelling component”). The term “aqueous medium” is to be understood in this context to include water, and liquids which are, or which approximate to, those present in the gastrointestinal tract of a mammal. Such polymer systems typically comprise hydrophilic macromolecular structures, which in a dry form may be in a glassy, or at least partially crystalline, state, and which swell when contacted with aqueous media. Modified release of drug is thus effected by one or more of the following processes: transport of solvent into the polymer matrix, swelling of the polymer, diffusion of drug through the swollen polymer and/or erosion of the polymer, one or more of which may serve to release drug slowly from the polymer matrix into an aqueous medium.

Thus, suitable polymeric materials (i.e. carriers), which may be used as the hydrophilic gelling component of a gelling matrix modified-release composition include those with a molecular weight of above 5000 g/mol, and which either:

(a) are at least sparingly soluble in; or

(b) swell when placed in contact with, aqueous media (as defined hereinbefore), so enabling release of drug from the carrier.

Suitable gelling matrix polymers, which may be synthetic or natural, thus s include polysaccharides, such as maltodextrin, xanthan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (e.g. poly(hydroxyethylmethacrylate)); carboxypolymethylene (e.g. Carbopol™); carbomer; polyvinylpyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; poly(ethylene oxide) (PEO); and cellulose ethers, such as hydroxymethylcellulose (HMC), hydroxyethylcellulose (HEC) hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na CMC); as well as copolymers and/or simple mixtures of any of the above polymers. Certain of the above-mentioned polymers may further be crosslinked by way of standard techniques.

For the compositions of the invention in the form of gelling matrix systems, we prefer that the principal swelling polymer that is employed is HPC, maltodextrin, scleroglucan or carboxypolymethylene, more preferably, PEO, HEC or xanthan, and, especially, HPMC, as well as copolymers and/or simple mixtures of any of these polymers.

When PEO, HEC, xanthan and HPMC are employed in (i.e. as at least one of the polymers of) the hydrophilic gelling component, preferred molecular weights (i.e. weight average molecular weights, as determined by standard techniques, such as osmometry, size-exclusion chromatography with a refraction detector (in which molecular weight is determined by way of standard calibration curves), light scattering and/or ultracentrifuge techniques), for these polymers are in the range 5,000 g/mol up to 200,000,000 g/mol, such as up to 100,000,000 g/mol, preferably up to 25,000,000 g/mol and more preferably up to 20,000,000 g/mol. Mixtures of PEO, HEC, xanthan and HPMC polymers with different molecular weights within these ranges may be employed.

Suitable HEC polymers also include those that produce solutions of polymer in water with viscosities, as measured by standard techniques, such as those described generally in the United States Pharmacopeia XXIV (USP XXIV/NF19) at page 2002 et seq (the relevant disclosures in which document are hereby incorporated by reference) of at least 200 cps for a 2% (w/w) aqueous solution and up to 8,000 cps for a 1% (w/w) aqueous solution, preferably at least 250 cps for a 2% aqueous solution and up to 5,500 cps for a 1% aqueous solution. Mixtures of HEC polymers with different viscosities within these ranges may be employed, in order, for example, to produce HEC mixtures which produce solutions as mentioned above with “average” viscosities (i.e. a viscosity for the mixture) within the above-mentioned preferred ranges. Similarly, mixtures of HEC polymers (with viscosities and/or “average” viscosities within these ranges) with other above-mentioned polymers may be employed. If HEC is employed as a polymer, it is preferred that the polymer is treated prior to tablet formulation, for example by way of milling and/or precipitating from acetone. Further, it may be desirable to coat a HEC polymer with another gelling polymer of a low viscosity (such as 6 cps HPMC), for example as described hereinafter. Suitable HEC polymers include those sold under the trademark NATRASOL™ (Aqualon).

Suitable HPMC polymers also include those that produce 2% w/w solutions of polymer in water with viscosities, as measured by standard techniques, such as those described generally in the United States Pharmacopeia XXIV (USP XXIV/NF19) at page 2002 et seq, as well as, specifically, at pages 843 and 844 (the relevant disclosures in which document are hereby incorporated by reference), of between 3 and 150,000 cps (at 20° C.), such as between 10 and 120,000 cps, preferably between 30 and 50,000 cps and more preferably between 50 and 15,000 cps. Mixtures of HPMC polymers with different viscosities within these ranges may be employed, in order, for example, to produce HPMC mixtures which produce solutions as mentioned above with “average” viscosities (i.e. a viscosity for the mixture) within the above-mentioned preferred ranges. Similarly, mixtures of HPMC polymers (with viscosities and/or “average” viscosities within these ranges) with other above-mentioned polymers may be employed. Suitable HPMC polymers include those fulfilling the United States Pharmacopeia standard substitution types 2208, 2906, 2910 and 1828 (see USP XXIV/NF19 for further details). Suitable HPMC polymers thus include those sold under the trademark METHOCEL™ (Dow Chemical Corporation) or the trademark METOLOSE™ (Shin-Etsu).

Suitable xanthan polymers include those that produce 1% w/w solutions of polymer in water with viscosities, as measured by standard techniques, such as those described generally in the United States Pharmacopeia XXIV (SP XXIV/NF19) at page 2002 et seq, as well as, specifically, at pages 2537 and 2538 (the relevant disclosures in which document are hereby incorporated by reference), of between 60 and 2,000 cps (at 24° C.), for example between 600 and 1,800 cps and preferably between 1,200 and 1,600 cps. Mixtures of xanthan polymers with different viscosities within these ranges may be employed, in order, for example, to produce xanthan mixtures which produce solutions as mentioned above with “average” viscosities (i.e. a viscosity for the mixture) within the above-mentioned preferred ranges. Similarly, mixtures of xanthan polymers (with viscosities and/or “average” viscosities within these ranges) with other above-mentioned polymers may be employed. Suitable xanthan polymers include those sold under the trademarks XANTURAL™ and KELTROL™ (CPKelco), and SATIAXANE™ (Degussa, Texturant Systems).

The choice of polymer will be determined by the nature of the active ingredient/drug (i.e. Compound A/B/C/D/salt) that is employed in the composition of the invention as well as the desired rate of release. In particular, it will be appreciated by the skilled person, for example in the case of HPMC, that a higher molecular weight will, in general, provide a slower rate of release of drug from the composition. Furthermore, in the case of HPMC, different degrees of substitution of methoxyl groups and hydroxypropoxyl groups will give rise to changes in the rate of release of drug from the composition. In this respect, and as stated above, it may be desirable to provide compositions of the invention in the form of gelling matrix systems in which the polymer carrier is provided by way of a blend of two or more polymers of, for example, different molecular weights, for example as described hereinafter, in order to produce a particular required or desired release profile.

When in the form of gelling matrix systems, we have also found that rate of release of drug from compositions of the invention may be further controlled by way of controlling the drug:polymer ratio within, and the surface area:volume ratio of, individual compositions (e.g. tablets) comprising drug and polymer carrier system.

Compositions of the invention, whether in the form of a gelling matrix system or otherwise, may contain one or more further excipients (in addition to the polymer carrier system) to further modify drug release, to improve the physical and/or chemical properties of the final composition, and/or to facilitate the process of manufacture. Such excipients are conventional in the formulation of modified release compositions.

For example, compositions of the invention may contain one or more of the following diluents: calcium phosphate (monocalcium phosphate, dicalcium phosphate and tricalcium phosphate), lactose, microcrystalline cellulose, mannitol, sorbitol, titanium dioxide, aluminium silicate and the like. Preferred diluents include microcrystalline cellulose.

Compositions of the invention may contain one or more of the following lubricants: magnesium stearate, sodium stearyl fumarate and the like.

Compositions of the invention may contain a glidant, such as a colloidal silica.

Compositions of the invention may contain one or more of the following binders: polyvinylpyrrolidone, lactose, mannitol, microcrystalline cellulose, a polyethylene glycol (PEG), a HPMC of a low molecular weight, a MC of a low molecular weight, a HPC of a low molecular weight and the like. Preferred binders include microcrystalline cellulose.

Compositions of the invention may contain one or more of the following pH controlling agents: organic acids (e.g. citric acid and the like) or alkali metal (e.g. sodium) salts thereof, pharmaceutically acceptable salts (e.g. sodium, magnesium or calcium salts) of inorganic acids (such as carbonic acid or phosphoric acid), oxides of magnesium, as well as alkali, and alkaline earth, metal (e.g. sodium, calcium, potassium and the like) sulphates, metabisulphates, propionates and sorbates.

Other furtler excipients may include colourants, flavourings, tonicity-modifying agents, coating agents, preservatives, etc.

Combinations of the above-stated further excipients may be employed.

It will be appreciated by the skilled person that some of the. above mentioned further excipients, which may be present in the final composition of the invention, may have more than one of the above-stated functions. Moreover, further excipients mentioned above may also function as part of a hydrophilic gelling component in a gelling matrix system.

The total amount of further excipients (not including, in the case of gelling matrix systems, the principal polymer carrier) that may be present in the composition of the invention will depend upon the nature of the composition, as well as the nature, and amounts of, the other constituents of that composition, and may be an amount of up to 85%, for example between 0.1 to 75%, such as 0.2 to 65%, preferably 0.3 to 55%, more preferably 0.5 to 45% and especially 1 to 40%, such as 2 to 35% w/w. In any event, the choice, and amount, of excipient(s) may be determined routinely (i.e. without recourse to inventive input) by the skilled person.

In gelling matrix systems, the amount of polymer in the system should be enough to ensure that a sufficient dose of drug is provided over the dosing interval to produce the desired therapeutic effect. Thus, we prefer that at least 60% (such as 80%) of the initial drug content of the composition is released to a patient, and/or under the test conditions described hereinafter, over a period of 2 hours or longer, preferably a period of 4 hours or longer, more preferably a period of 6 hours or longer and particularly over a period of between 8 and 24 hours. Suitable amounts of polymer that may be included, which will depend upon inter alia the active ingredient that is employed in the composition, any excipients that may be present and the nature of the polymer that is employed, are in the range 5 to 99.5%, for example 10 to 95%, particularly 15 to 80%, preferably 20 to 75%, more preferably 30 to 70% and especially 35 to 65% w/w. In any event, the choice, and amount, of polymer may be determined routinely by the skilled person.

When compositions of the invention are provided in the form of gelling matrix systems, active ingredients (Compounds A, B, C, D, or pharmaceutically-acceptable salts of any of those compounds) that may be mentioned-ilclude the free base forms of Compounds A, B, C and, especially, D, as well as salts in which the solubility of that salt in aqueous media (as defined above) is substantially independent of the pH of that medium, particularly pHs in the physiological range typically found in the gastrointestinal tract.

Preferred salts of Compound A thus include 1-hydroxy-2-naphthoic acid salts, benzoic acid salts, 2-mesitylenesulphonic acid salts, hydroxy-substituted benzenesulphonic acid salts, 1,5-naphthalenesulphonic acid salts, 1,5-naphthalenedisulphonic acid salts, particularly, toluenesulphonic acid salts, or, especially, benzenesulphonic acid salts.

Preferred salts of Compounds B, C and D may thus include methanesulphonic acid salts, hippuric acid salts, toluenesulphonic acid salts, pamoic acid salts, 1,5-naphthalenedisulphonic acid salts, terephthalic acid salts, succinic acid salts, salts of tartaric acid and derivatives thereof, such as O,O′-dibenzoyltartaric acid salts and O,O′-di-para-toluoyltartaric acid salts, 2,2,3,3-tetramethyl-1,4-dibutanoic acid salts, 1,2-cyclopentanedi-carboxylic acid salts, or acid addition salts in which the acid is a derivative of hippuric acid, for example an acid of formula I,

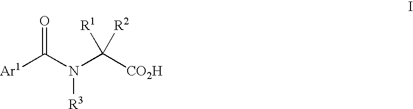

wherein

Ar¹ represents phenyl or naphthyl, both of which are optionally substituted by one or more substituents selected from halo (e.g. chloro), nitro, C₁₋₆ alkyl, C₁₋₆ alkoxy, hydroxy and phenyl; and

R¹, R² and R³ independently represent H or C₁₋₃ alkyl.

It will be appreciated by the skilled person that when Ar¹ represents phenyl and R¹, R² and R³ all represent H, then the acid of formula I is hippuric acid.

Preferred Ar¹ groups include phenyl, which phenyl group is optionally substituted by phenyl (for example in the 4-position relative to the point of attachment of the C(O) group), chloro (for example in the 3- and/or 4-positions relative to the C(O) group), nitro (for example in the 4-position relative to the C(O) group) and/or C₁₋₄ alkyl, such as methyl (for example in the 2- and/or 4-positions relative to the C(O) group); and naphthyl. More preferred values of Ar¹ include phenyl, 4-phenylphenyl (biphenyl), 3,4-dichlorophenyl, 2-naphthyl, 4-nitrophenyl and 2,4,6-trimethylphenyl.

Preferred R¹ and R² groups include H and methyl. It is preferred that R¹ and R² either both represent H or both represent methyl.

Preferred R³ groups include H.

When R¹ and R² both represent methyl, it is preferred that Ar¹ represents phenyl. When R¹ and R² both represent H, it is preferred that Ar¹ represents 4-nitrophenyl, 2,4,6-trimethylphenyl or, especially, 3,4-dichlorophenyl, 2-naphthyl or 4-phenylphenyl (biphenyl).

Acids of formula I are commercially available (e.g. hippuric acid, 4-nitrohippuric acid and 2-, 3- or 4-methylhippuric acid), or may be prepared in accordance with standard techniques.

For example acids of formula I may be prepared by reaction of a compound of formula II,

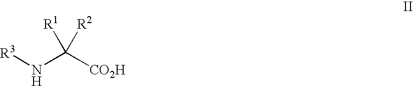

wherein R¹, R² and R³ are as hereinbefore defined, with an acid chloride of formula III,

Ar¹(C(O)Cl III

wherein Ar¹ is as hereinbefore defined, for example in the presence of base, e.g. aqueous NaOH, in accordance with classical Schotten-Baumann procedures (see, for example, J. Med. Chem., 1989, 32, 1033).

Neutralisation with acid, e.g. conc. hydrochloric acid, may precipitate the acid of formula I, which may be recrystallised if necessary from various solvents, e.g. iso-propyl alcohol, methanol, ethanol, acetone and water, or mixtures of those solvents.

Alternatively, ester (e.g. lower alkyl ester) derivatives of compounds of formula II, optionally in the form of a salt, e.g. the hydrochloride salt, can be reacted with an acid chloride of formula m, in the presence of base, e.g. triethylamine, in a suitable solvent, e.g. dichloromethane, to give an ester-amide of formula IV,

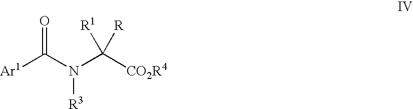

wherein R⁴ represents lower alkyl (such as C₁₋₆ alkyl) or lower alkylphenyl (e.g. C₁₋₃ alkylphenyl) and Ar¹, R¹, R² and R³ are as hereinbefore defined (see, for example, J. Heterocyclic Chem. 1973, 10, 935, Tetrahedron 1989, 45, 1691 and J. Org. Chem., 1999, 64, 8929). Ester-amides of formula IV may be solids at room temperature and may thus be purified by crystallisation following their formation, if appropriate. Compounds of formula IV may then be converted to compounds of formula I by standard hydrolysis, e.g. with aqueous sodium hydroxide followed by addition of an acid, e.g. hydrochloric acid, to precipitate the product. Recrystallisation may then be carried out, if required.

Compounds of formulae I, II and IV in which R³ represents C₁₋₃ alkyl may be made by standard alkylation of a corresponding compound of formula I, II or IV in which R³ represents H.

Compounds of formulae II (and ester derivatives) and III are commercially available or may be made readily by way of routine techniques.

Preferred salts of Compound D include methanesulphonic acid, pamoic acid, 1,5-naphthalenedisulphonic acid, hippuric acid, terephthalic acid, succinic acid, O,O′-dibenzoyl-D-tartaric acid, O,O′-di-para-toluoyl-D-tartaric acid, 2,2,3,3-tetramethyl-1,4-dibutanoic acid and 1,2-cyclopentanedicarboxylic acid salts, and acid addition salts in which the acid is a compound of formula I as hereinbefore defined, for example 4-phenylhippuric acid, (3,4-dichlorobenzoylamino)acetic acid and [(naphthalene-2-carbonyl)amino]acetic acid salts. Particularly preferred salts of Compound D include methanesulphonic acid salts.

Preferred salts of Compound C include methanesulphonic acid salts and toluenesulphonic acid salts e.g. para-toluenesulphonic acid salts.

Preferred active ingredients for use in the compositions of the invention, and especially gelling matrix systems, include Compound D and pharmaceutically acceptable salts thereof, particularly Compound D in the form of the free base or in the form of a methanesulphonic acid salt.

Suitable amounts of active ingredient in the compositions of the invention, whether in the form of gelling matrix systems or otherwise, depend upon many factors, such as the nature of that ingredient (free base/salt etc), the dose that is required, and the nature, and amounts, of other constituents of the composition. However, they may be in the range 0.5 to 80%, for example 1 to 75%, such as 3 to 70%, preferably 5 to 65%, more preferably 10 to 60% and especially 15 to 55% w/w. In any event, the amount of active ingredient to be included may be determined routinely by the skilled person.

Typical daily doses of Compounds A, B, C or D, or pharmaceutically-acceptable salts of any of these compounds, are in the range 10 to 2000 mg, e.g. 25, such as 30, to 1200 mg of free base (i.e., in the case of a salt, excluding any weight resulting from the presence of a counter ion), irrespective of the number of compositions (e.g. tablets) that are administered during the course of that day. Preferred daily doses are in the range 50 to 1000 mg, such as 100 to 500 mg. Typical doses in individual compositions of the invention (e.g. tablets) are thus in the range 15 to 500 mg, for example 40 to 400 mg.

Compositions of the invention such as those described hereinbefore may be made in accordance with well known techniques such as those described in the references mentioned hereinbefore. Compositions of the invention that are in the form of gelling matrix systems may be prepared by standard techniques, and using standard equipment, known to the skilled person, including wet or dry granulation, direct compression/compaction, drying, milling, mixing, tabletting and coating, as well as combinations of these processes, for example as described hereinafter.

Although compositions of the invention are preferably adapted to be administered orally, their use is not limited to that mode of administration. Parenteral modified release compositions of the invention, which may include systems that are well known to those skilled in the art, such as those based upon poloxamers, biodegradable microspheres, liposomes, suspensions in oils and/or emulsions, may be prepared in accordance with standard techniques, for example as described by Leung et al in “Controlled Drug Delivery: Fundamentals and Applications” (Drugs and the Pharmaceutical Sciences; vol. 29), 2^{nd} edition, eds. Robinson and Lee, Dekker (1987) at Chapter 10, page 433, the disclosure in which document is hereby incorporated by reference.

The compositions of the invention may be dosed once or more times daily (e.g. up to six times, but preferably no more than twice, daily), irrespective of the number of individual units (formulations/compositions) that are administered as part of one “dose”.

The compositions of the invention are useful in the delivery of Compounds A, B, C, D and pharmaceutically-acceptable salts thereof to patients. As Compounds A, B, C, D and pharmaceutically-acceptable salts thereof are useful in both the prophylaxis and the treatment of cardiac arrhythmias, in particular atrial and ventricular arrhythmias (such as atrial fibrillation (e.g. atrial flutter)), the compositions of the invention are also expected to be useful in the treatment of such disorders.

The compositions of the invention are thus indicated in the treatment or prophylaxis of cardiac diseases, or in indications related to cardiac diseases, in which arrhythmias are believed to play a major role, including ischaemic heart disease, sudden heart attack, myocardial infarction, heart failure, cardiac surgery and thromboembolic events.

According to a further aspect of the invention, there is provided a method of treatment of an arrhythmia which method comprises administration of a composition of the invention to a person suffering from, or susceptible to, such a condition.

For the avoidance of doubt, by “treatment” we include the therapeutic treatment, as well as the prophylaxis, of a condition.

Compositions of the invention have the advantage that they may provide a modified release of Compounds A, B, C, D or a pharmaceutically-acceptable salt of any of these compounds, in order to obtain a more even and/or prolonged effect against cardiac arrhythmias and may thus provide efficient dosing of active ingredient preferably no more than once or twice daily. Certain compositions of the invention may achieve this release in an essentially pH-independent manner.

Compositions of the invention may also have the advantage that they may be prepared using established pharmaceutical processing methods and employ materials that are approved for use in foods or pharmaceuticals or of like regulatory status.

The invention is illustrated, but in no way limited, by the following examples, in which:

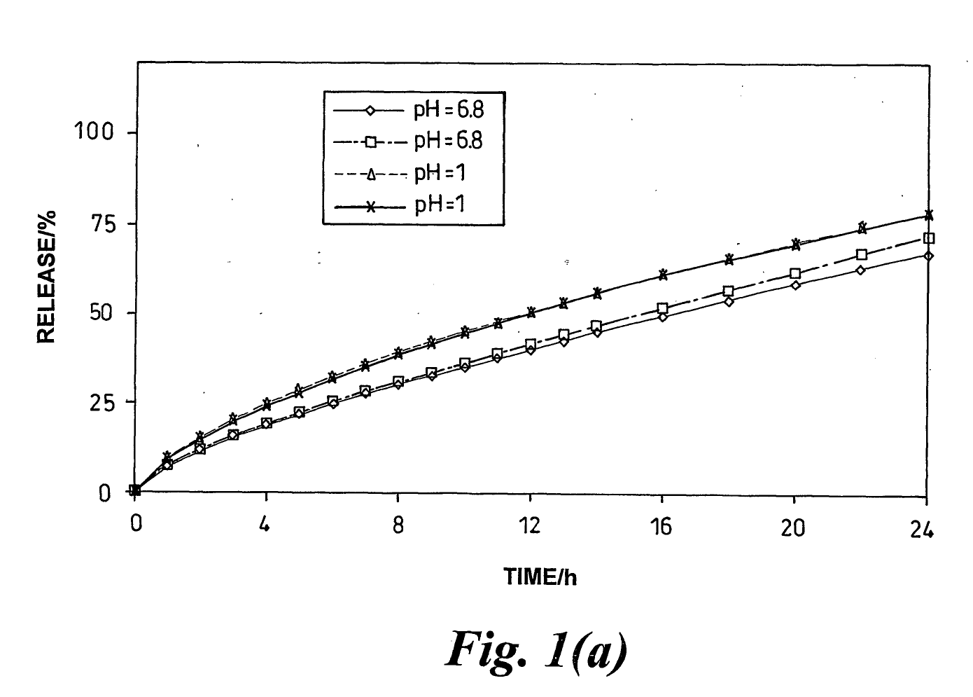

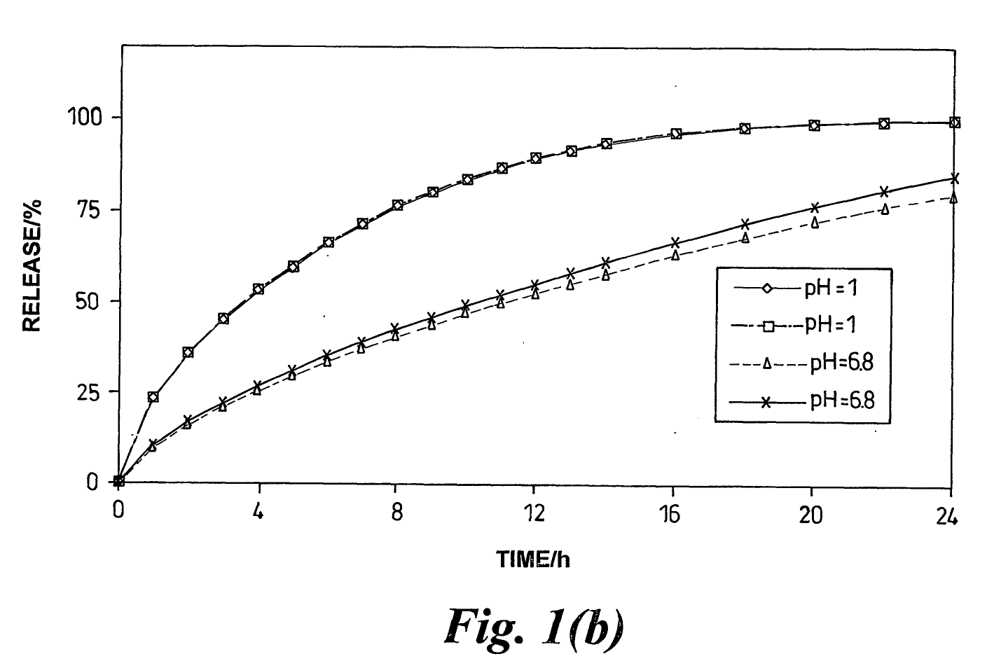

FIG. 1(a) shows the drug release profile (scaled to 100%) at different pHs of the benzenesulphonate salt of Compound A from tablets made from a specific grade of HPMC polymer (METOLOSE™ 65SH1500; Shin-Etsu).

FIG. 1(b) shows the drug release profile (scaled to 100%) at different pHs of Compound A in the form of the free base from tablets made from a specific grade of HPMC polymer (METOLOSE™ 65SH1500; Shin-Etsu).

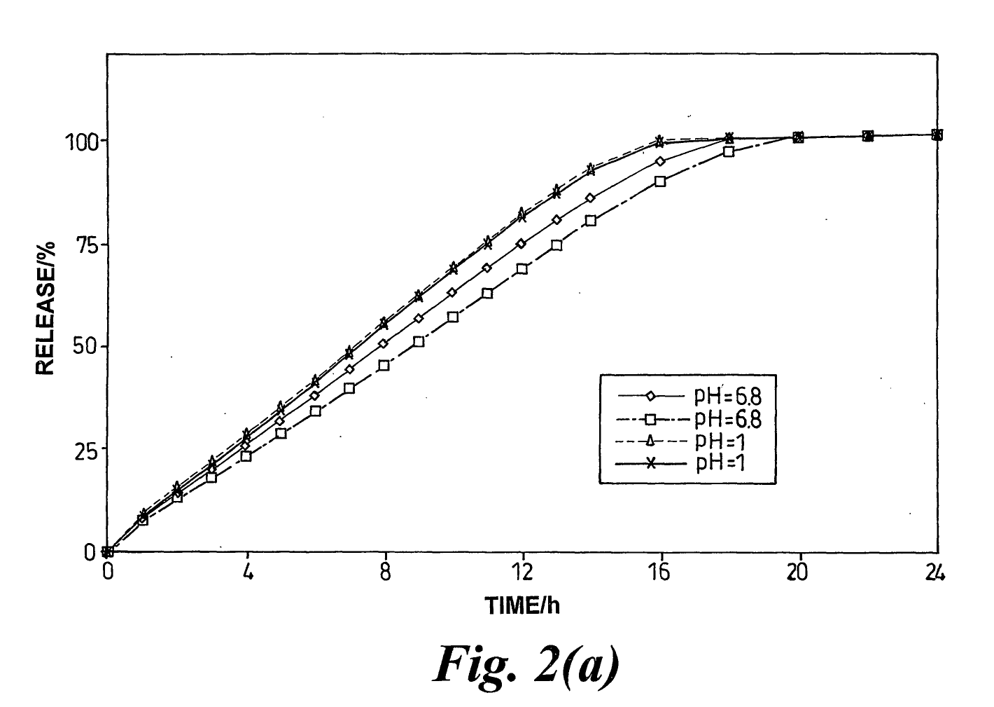

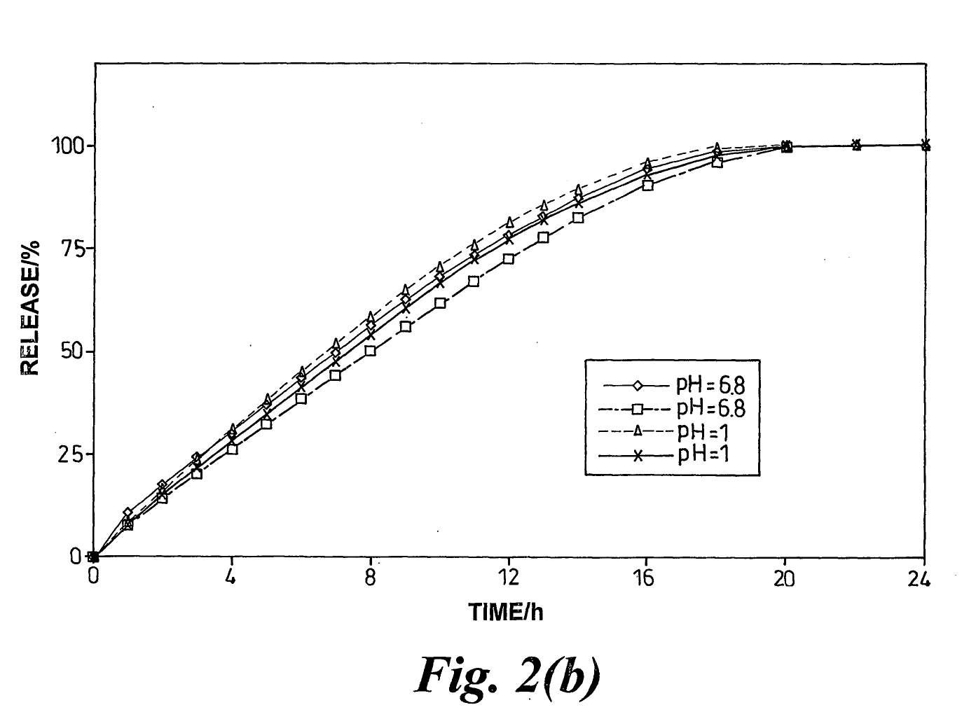

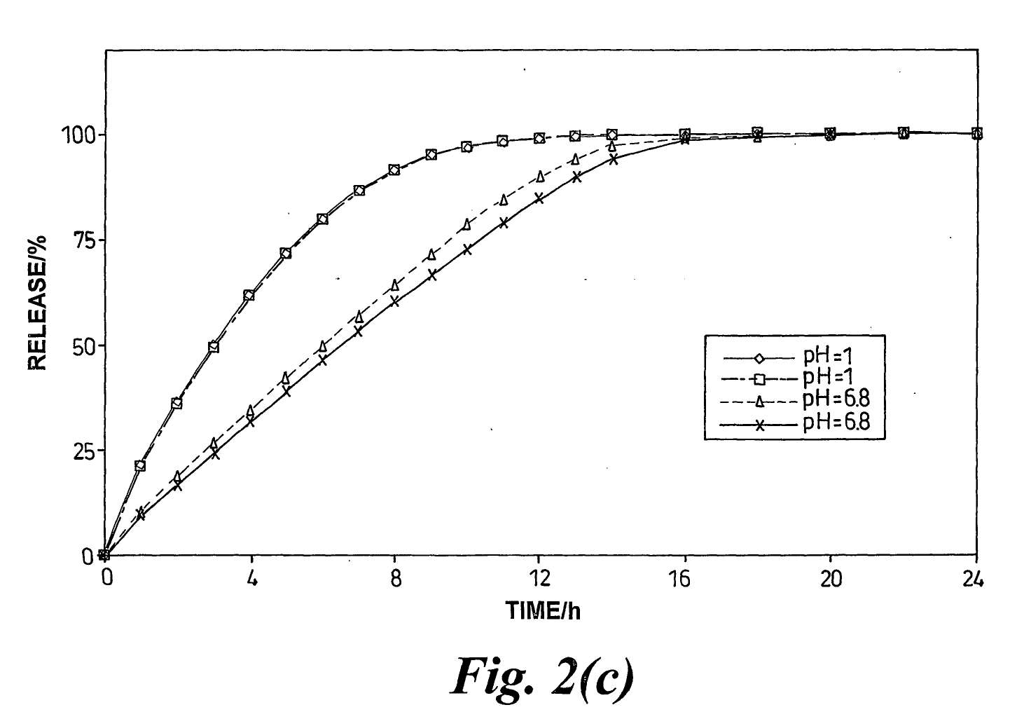

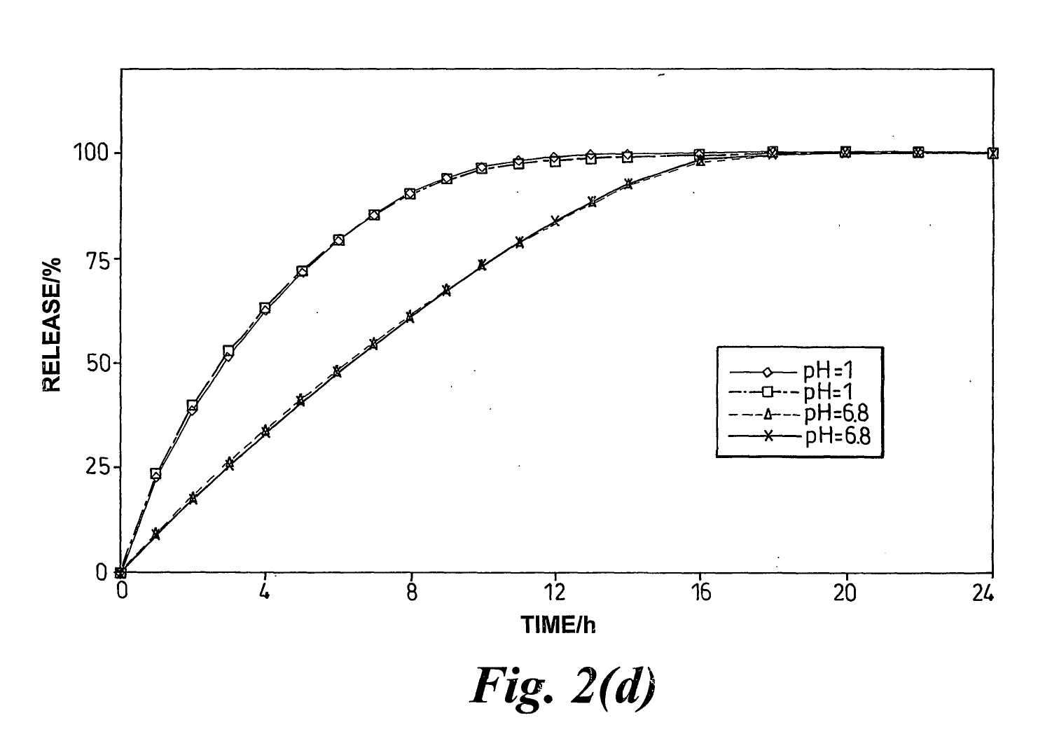

FIG. 2(a) shows the drug release profile (scaled to 100%) at different pHs of the benzenesulphonate salt of Compound A from tablets made from a specific grade of PEO polymer (molecular weight 4×10⁶ g/mol).

FIG. 2(b) shows the drug release profile (scaled to 100%) at different pHs of the benzenesulphonate salt of Compound A from tablets made from a specific grade of HEC polymer (NATRASOL® 250M Pharm).

FIG. 2(c) shows the drug release profile (scaled to 100%) at different pHs of Compound A in the form of the free base from tablets made from a specific grade of PEO polymer (molecular weight 4×10⁶ g/mol).

FIG. 2(d) shows the drug release profile (scaled to 100%) at different pHs of Compound A in the form of the free base from tablets made from a specific grade of HEC polymer (NATRASOL® 250M Pharm).

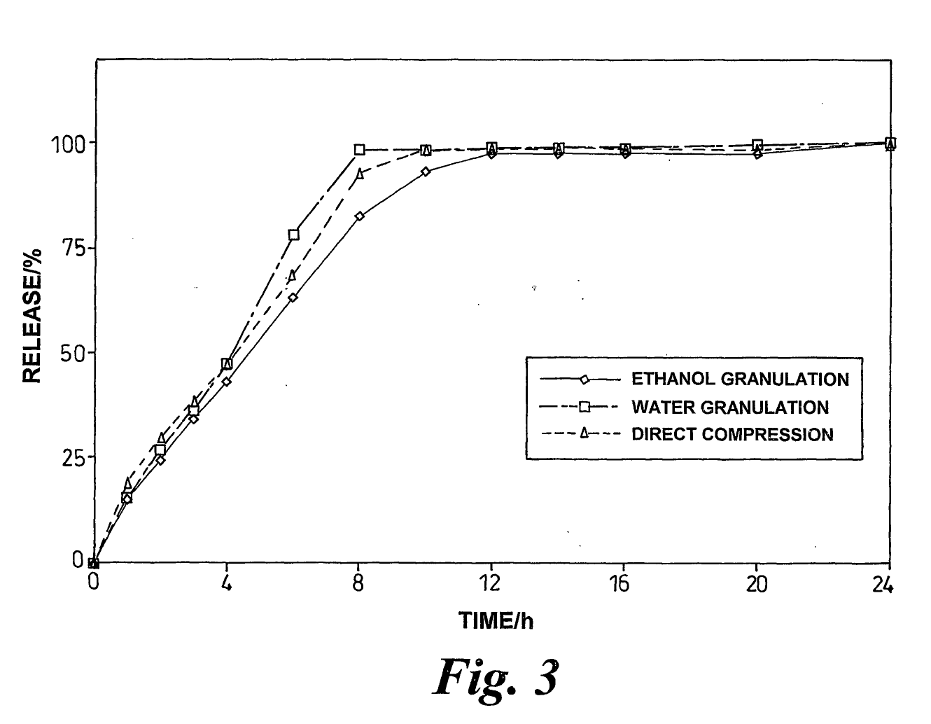

FIG. 3 shows the drug release profile (scaled to 100%) at pH 6.8 of the benzenesulphonate salt of Compound A from tablets made via different processes from a specific grade of HPMC polymer (METOLOSE™ 65SH400; Shin-Etsu).

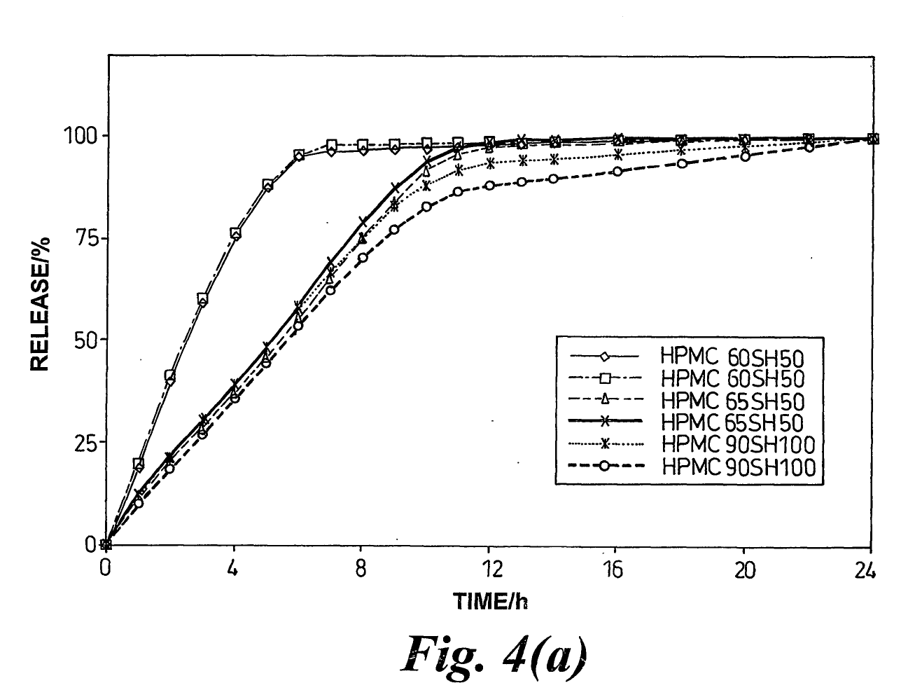

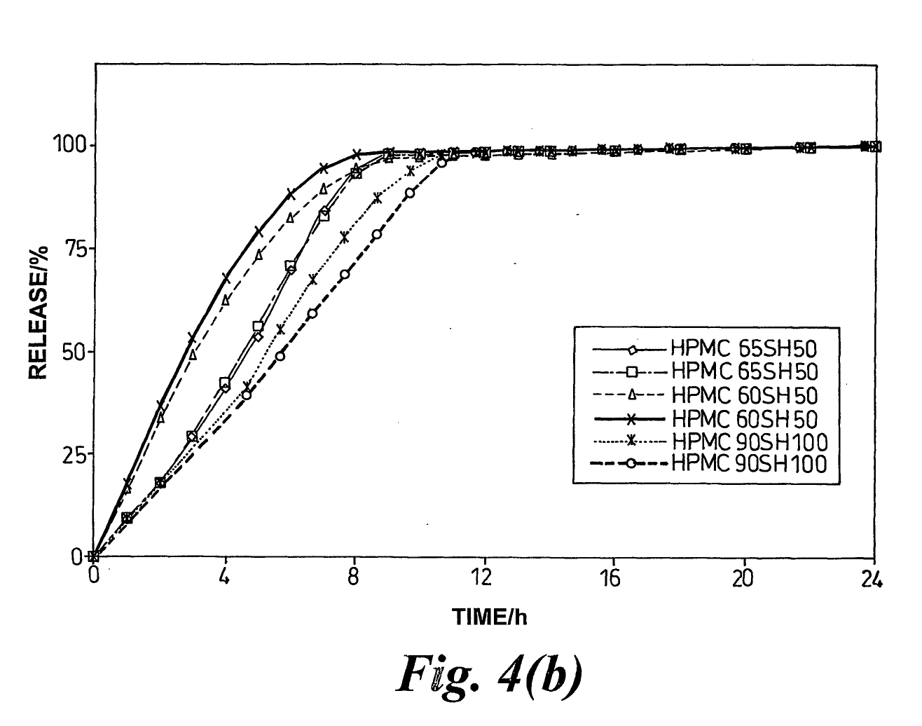

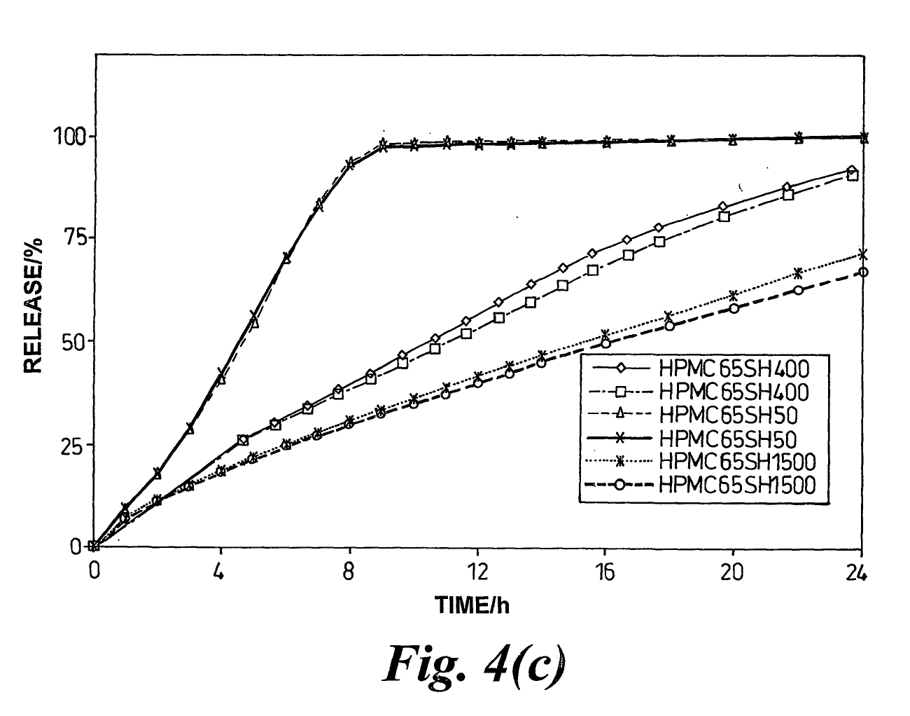

FIG. 4(a) shows the drug release profile (scaled to 100%) at pH 1.0 of the benzenesulphonate salt of Compound A from tablets made from three specific grades of HPMC polymer with different degrees of substitution (METOLOSE™ 60SH50, METOLOSE™ 65SH50 and METOLOSE™ 90SH100; Shin-Etsu).

FIG. 4(b) shows the drug release profile (scaled to 100%) at pH 6.8 of the benzenesulphonate salt of Compound A from tablets made from three specific grades of HPMC polymer with different degrees of substitution (METOLOSE™ 60SH50, METOLOSE™ 65SH50 and METOLOSE™ 90SH100; Shin-Etsu).

FIG. 4(c) shows the drug release profile (scaled to 100%) at pH 6.8 of the benzenesulphonate salt of Compound A from tablets made from three specific grades of HPMC polymer with different molecular weights (METOLOSE™ 65SH400, METOLOSE™ 65SH50 and METOLOSE™ 65SH1500; Shin-Etsu).

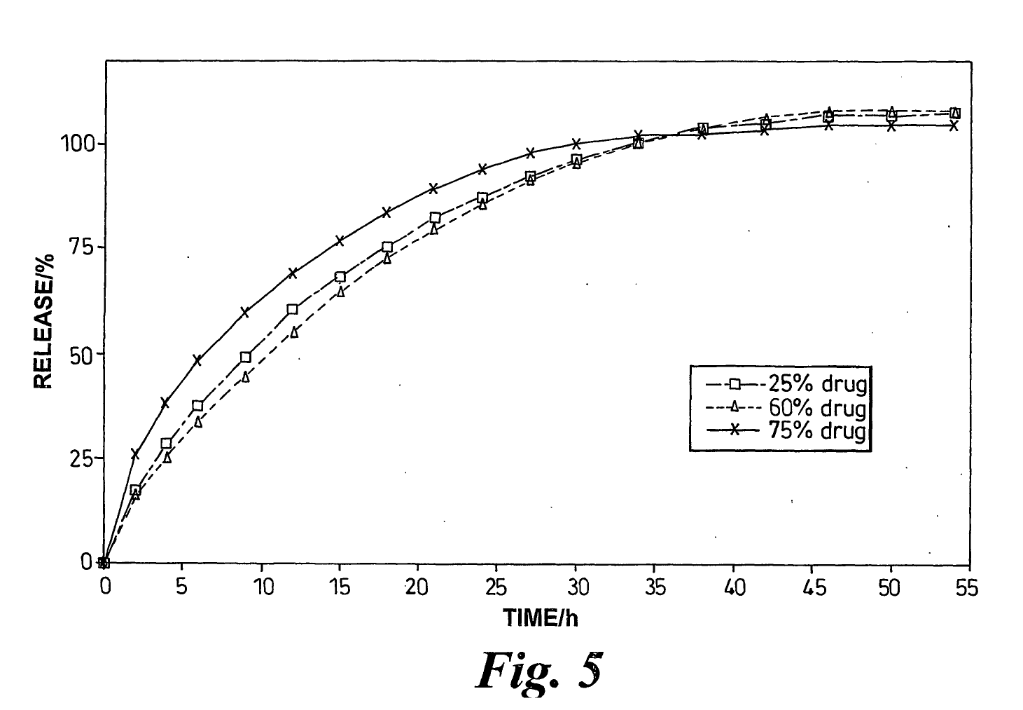

FIG. 5 shows the drug release profile at pH 6.8 of the benzenesulphonate salt of Compound A from tablets made from a specific grade of HPMC polymer (METOLOSE™ 60SH10000; Shin-Etsu), in which the tablets comprise different drug: polymer ratios.

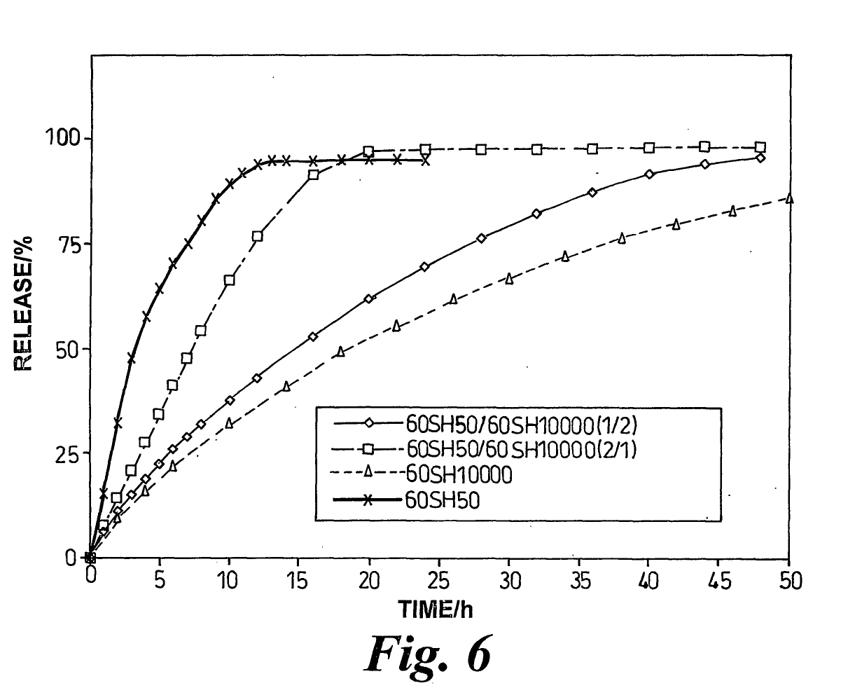

FIG. 6 shows the drug release profile at pH 6.8 of the benzenesulphonate salt of Compound A from tablets made from specific grades of HPMC polymer (METOLOSE™ 60SH50 and METOLOSE™ 60SH10000; Shin-Etsu), either alone or dry mixed together in different weight ratios.

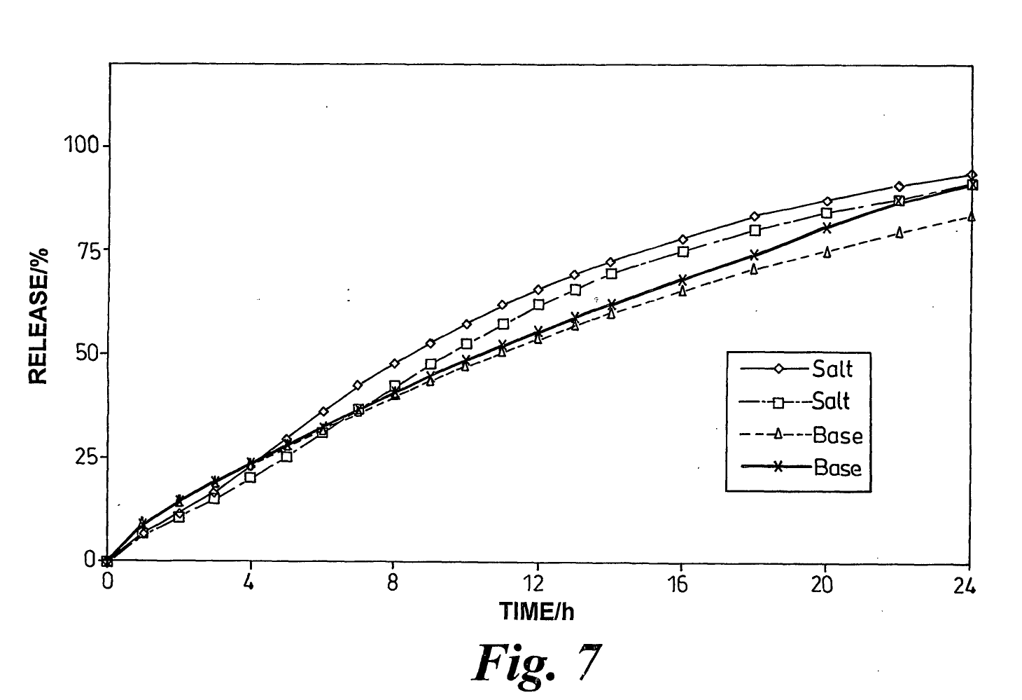

FIG. 7 shows the drug release profile (scaled to 100%) at pH 6.8 of Compound A in the form of the free base and as the benzenesulphonate salt thereof from tablets made from a specific grade of HPMC polymer (METOLOSE™ 65SH1500; Shin-Etsu).

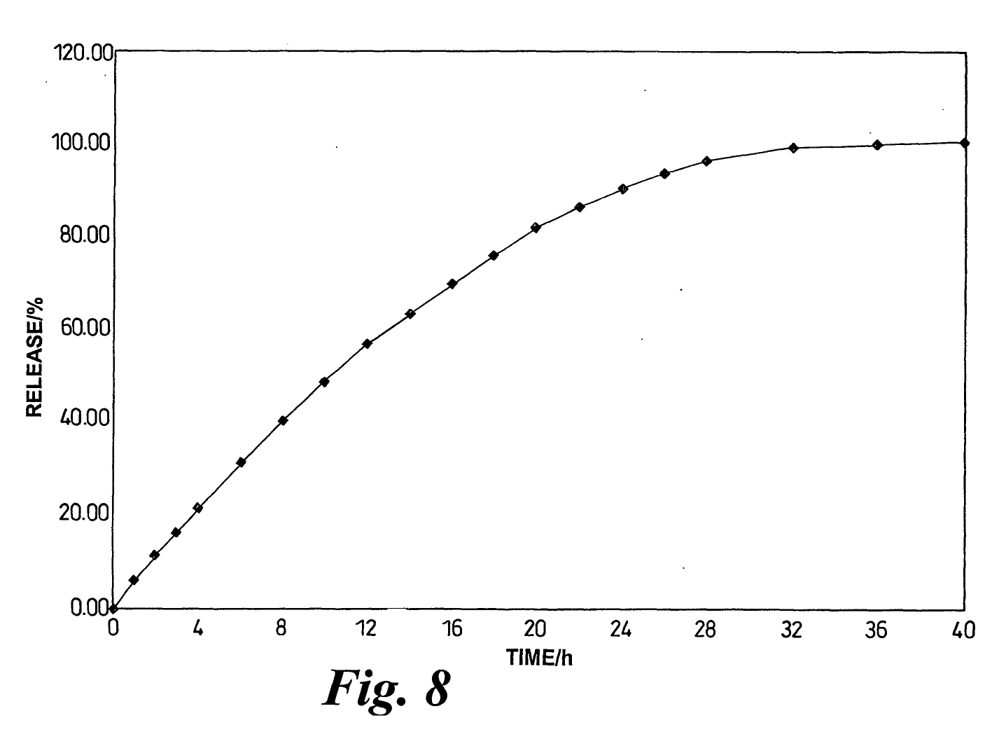

FIG. 8 shows the drug release profile at pH 6.8 of benzenesulphonate salt of Compound A from tablets made from a blend of specific grades of HPMC polymers (METHOCEL™ KlOOLV CR and METHOCEL™ K4M; Dow) (average of six tablets).

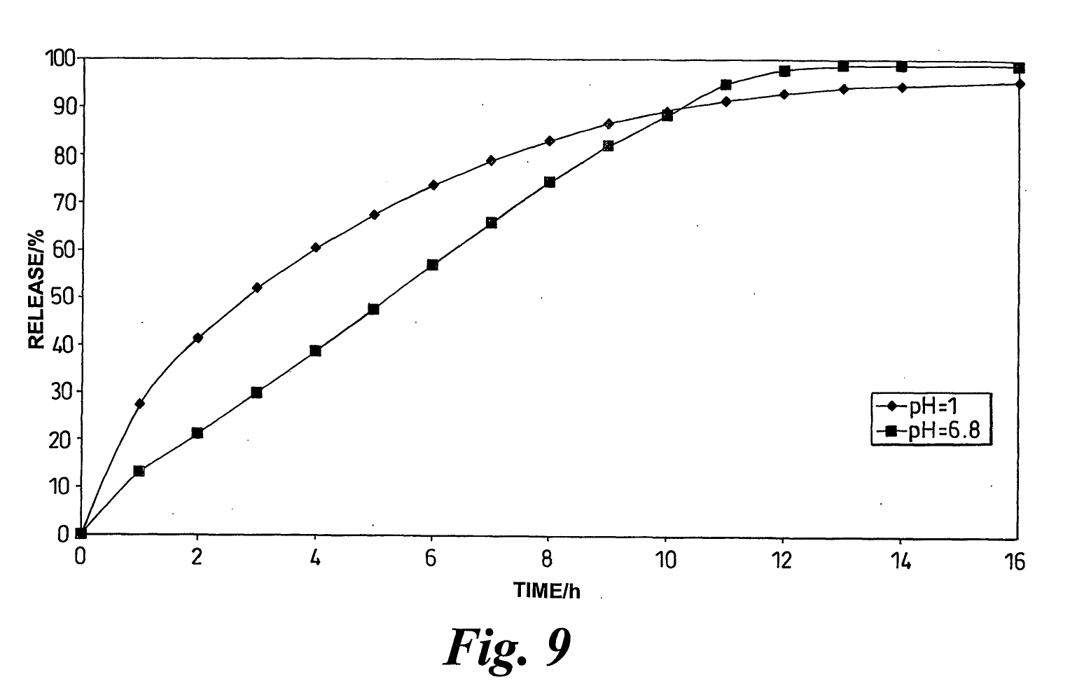

FIG. 9 shows the drug release profile at different pHs of Compound D (free base) from tablets made from a specific grade of HPMC polymer (METOLOSE™ 65SH50; Shin-Etsu).

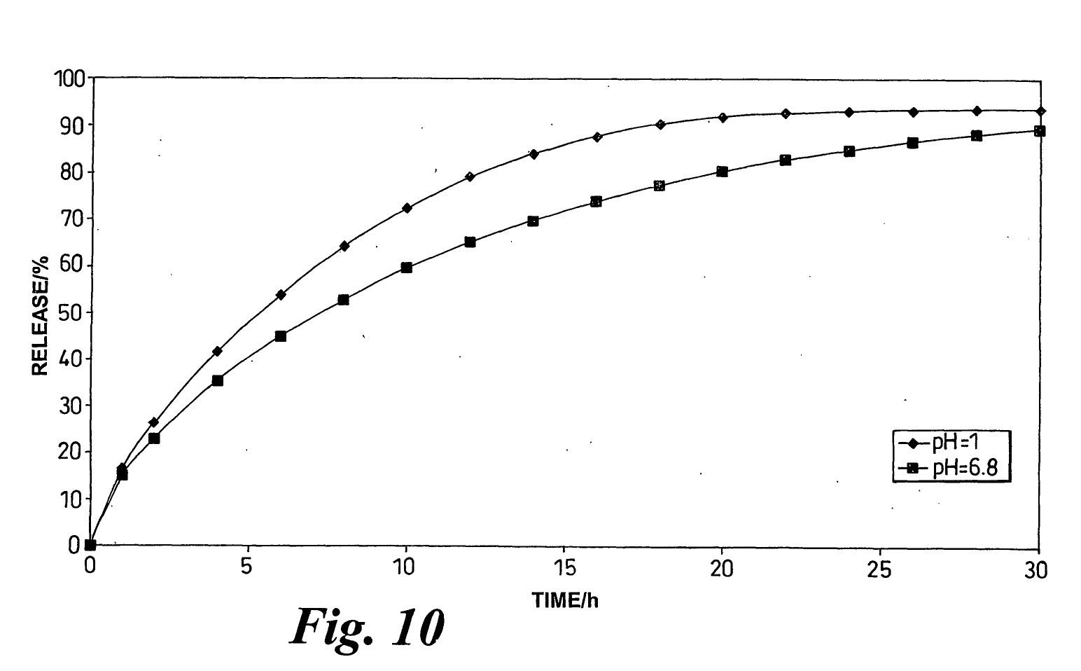

FIG. 10 shows the drug release profile at different pHs of Compound D (free base) from tablets made from a blend of specific grades of HPMC polymers (METHOCEL™ 60SH50 and METHOCEL™ 60SH10000; Shin-Etsu).

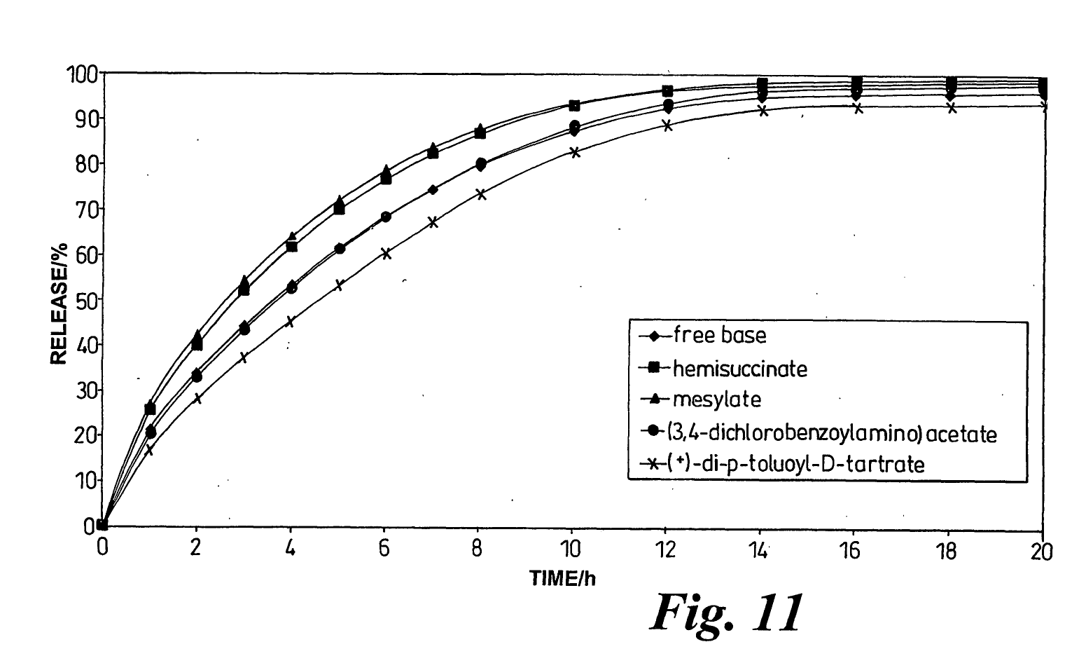

FIG. 11 shows the drug release profile at pH 6.8 of Compound D (free base and various salts thereof) from tablets made from a blend of specific grades of HPMC polymers (METHOCEL™ 60SH50 and METHOCEL™ 60SH10000; Shin-Etsu).

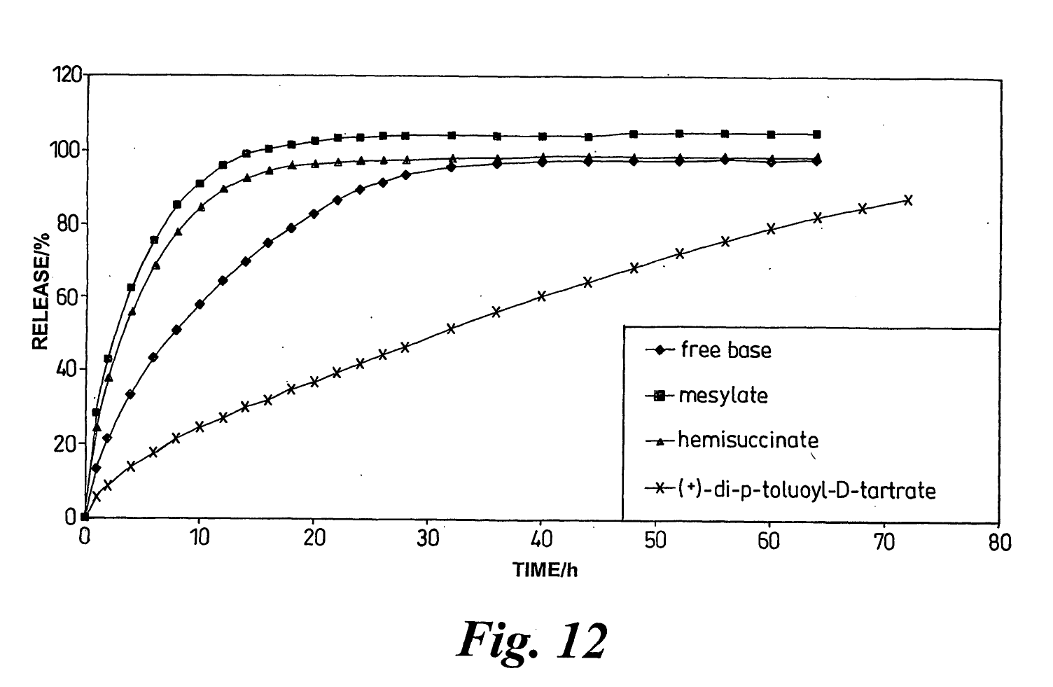

FIG. 12 shows the drug release profile at pH 6.8 of Compound D (free base and various salts thereof) from tablets made from a specific grade of HPMC polymer (METHOCEL™ 60SH10000; Shin-Etsu).

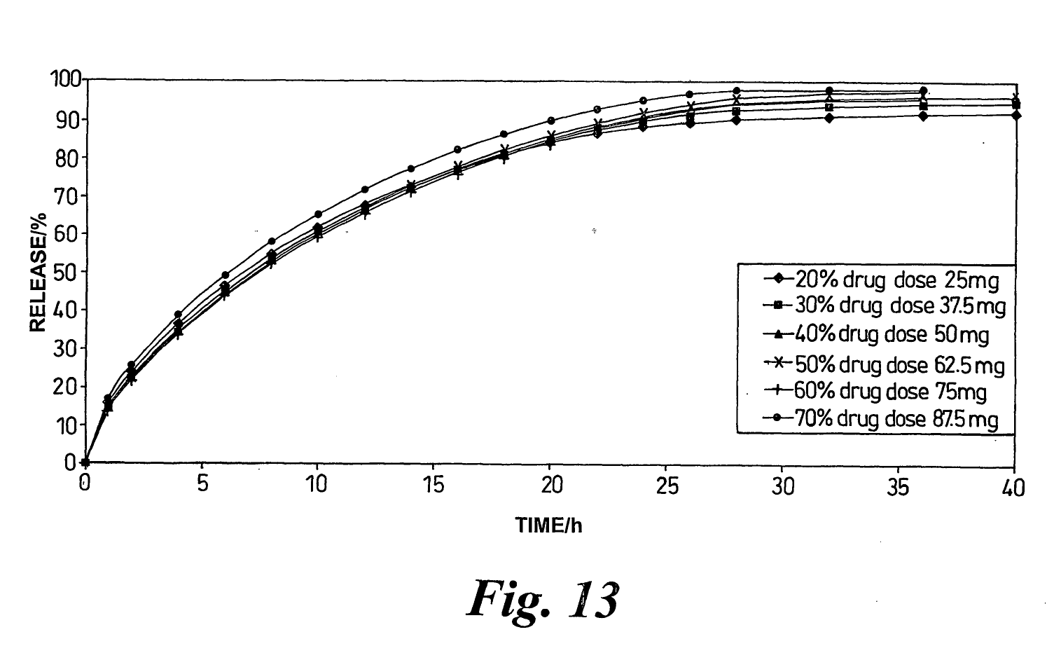

FIG. 13 shows the drug release profile at pH 6.8 of Compound D (free base) from tablets made from a specific grade of HPMC polymer (METHOCEL™ 60SH10000; Shin-Etsu), in which the tablets comprise different drug:polymer ratios (8 mm tablet size; 125 mg tablet weight; different doses of drug).

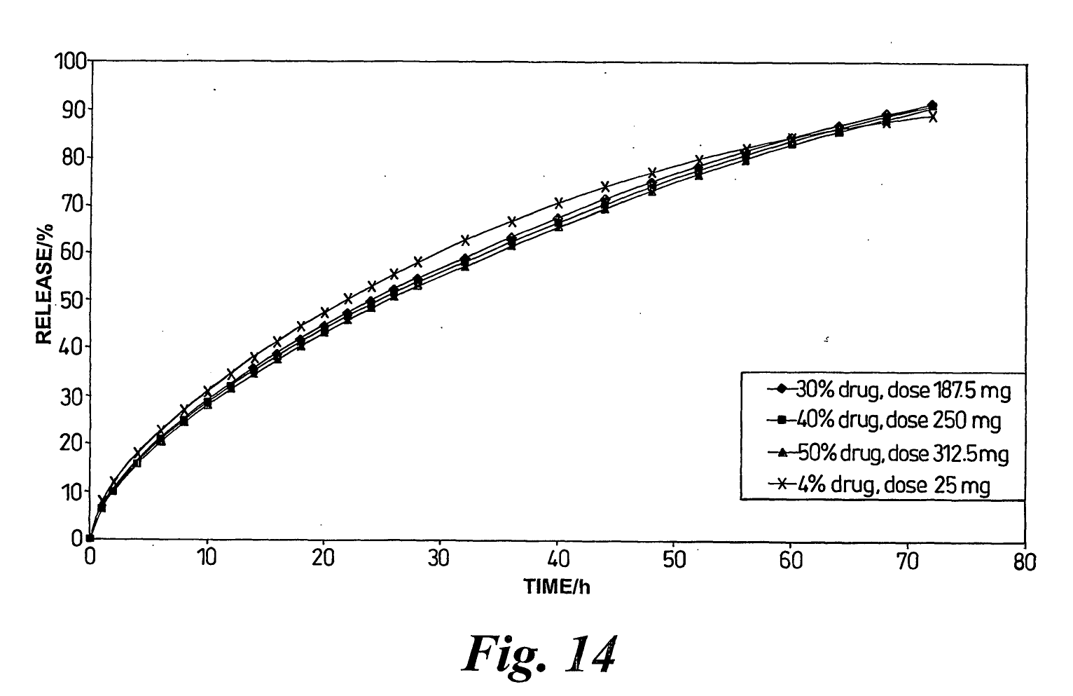

FIG. 14 shows the drug release profile at pH 6.8 of Compound D (free base) from tablets made from a specific grade of HPMC polymer (METHOCEL™ 60SH10000; Shin-Etsu), in which the tablets comprise different drug:polymer ratios (12 mm tablet size; 625 mg tablet weight; different doses of drug).

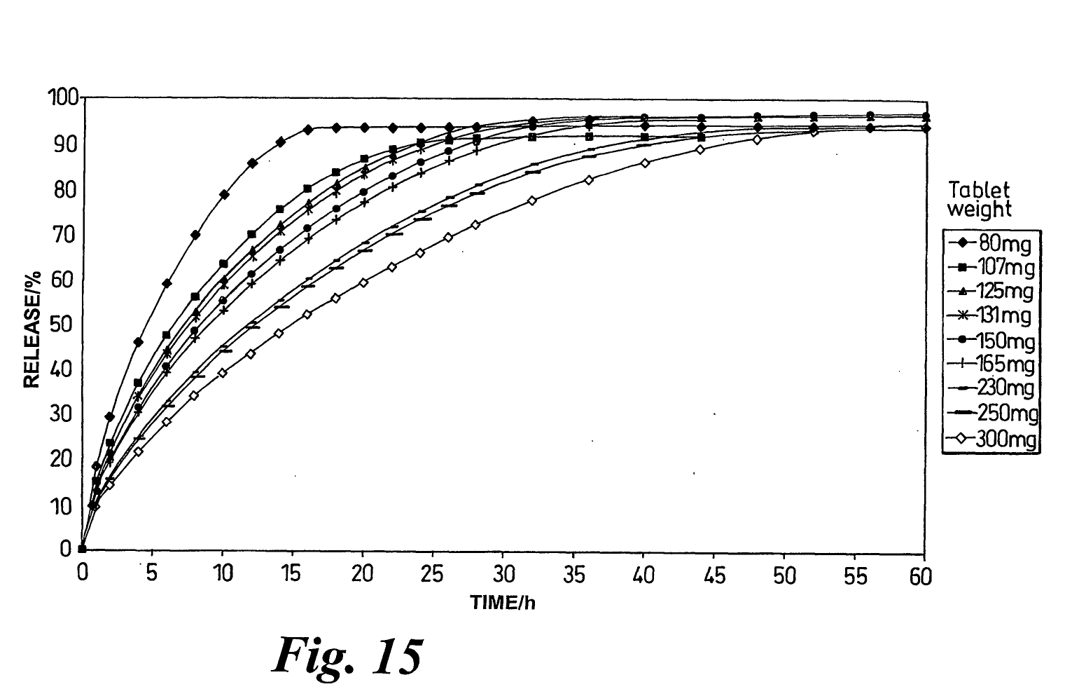

FIG. 15 shows the drug release profile at pH 6.8 of Compound D (free base) from tablets made from a specific grade of HPMC polymer (METHOCEL™ 60SH10000; Shin-Etsu), in which the tablets comprise different drug:polymer ratios (8 mm tablet size; different tablet weights; same dose of drug).

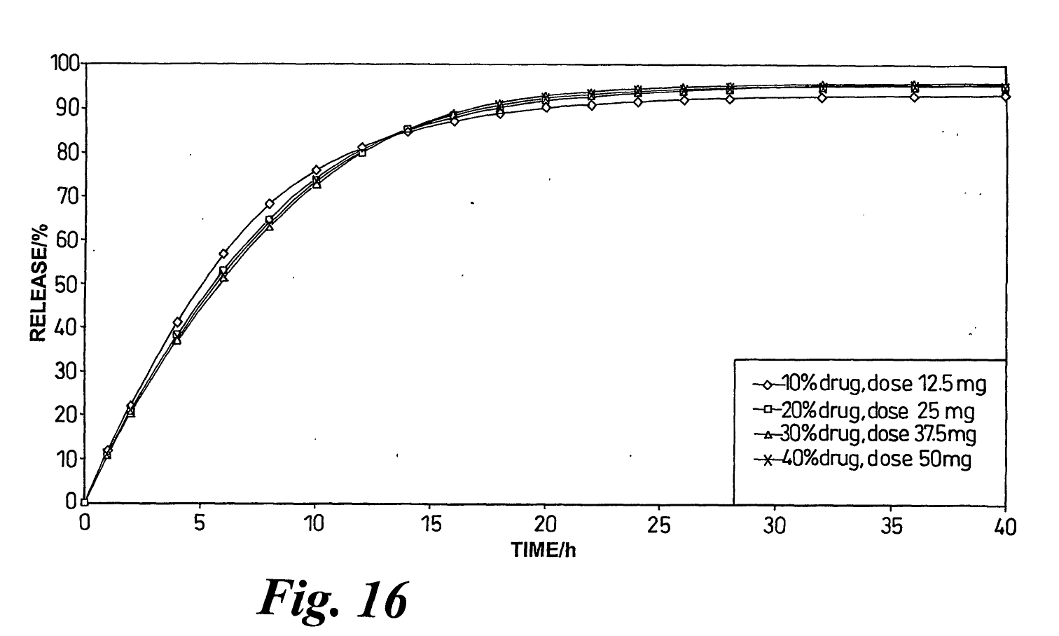

FIG. 16 shows the drug release profile at pH 6.8 of Compound D (free base) from tablets made from a specific grade of xanthan gum (XANTURAL® 180; CPKelco) in which the tablets comprise different drug:polymer ratios (8 mm tablet size; 125 mg tablet weight; different doses of drug).

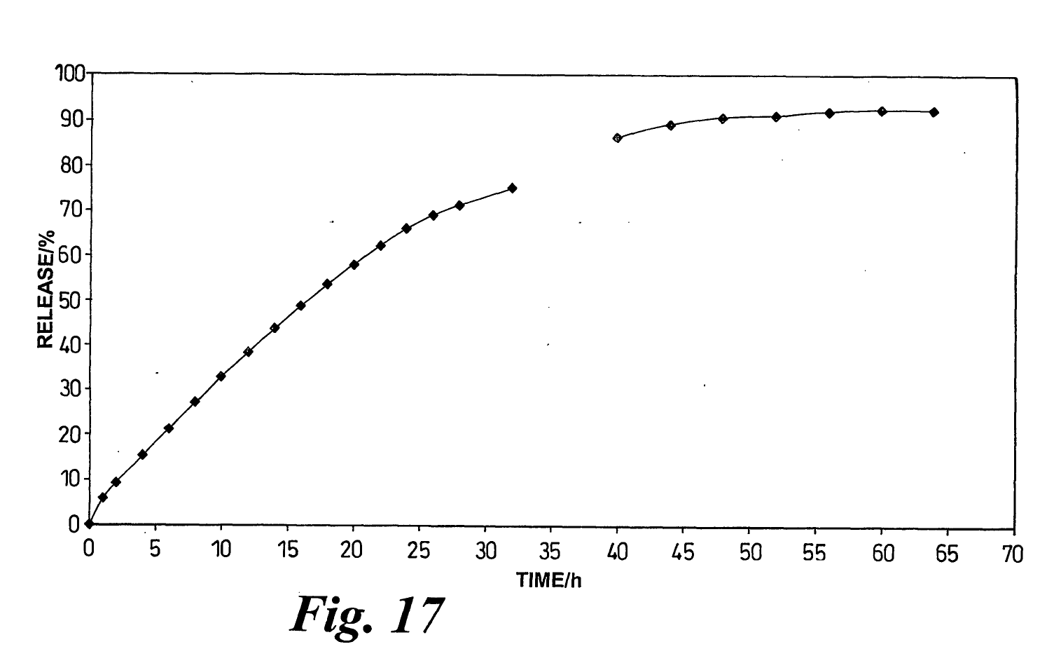

FIG. 17 shows the drug release profile at pH 6.8 of Compound D (free base) from tablets made from a specific grade of xanthan gum (KELTROL®D; CPKelco).

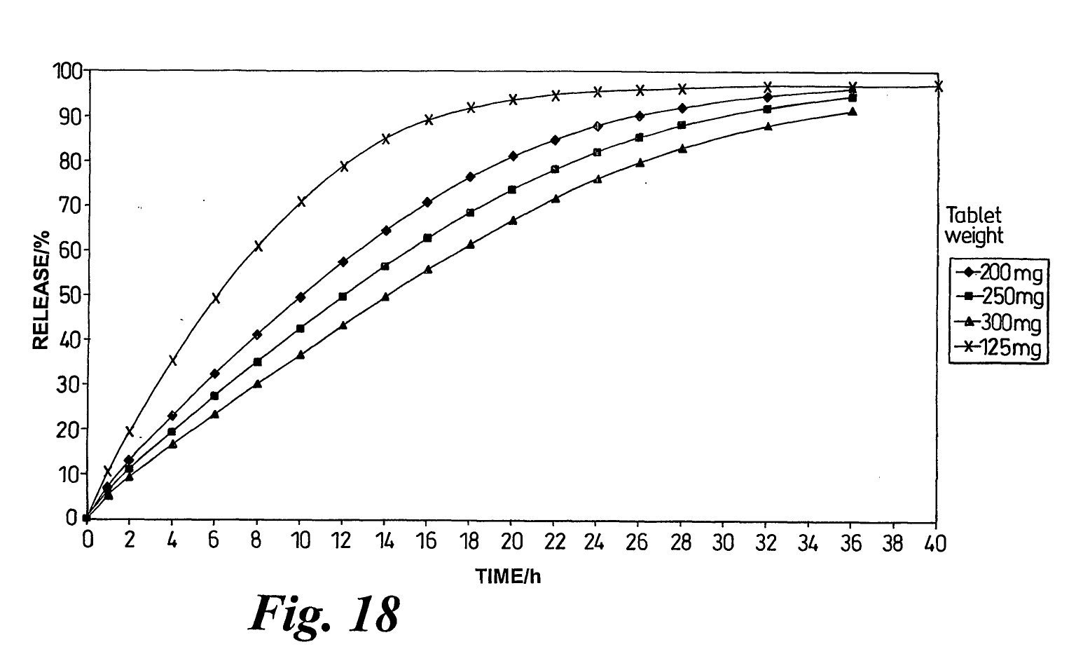

FIG. 18 shows the drug release profile at pH 6.8 of Compound D (free base) from tablets made from a specific grade of xanthan gum (XANTURAL® 180; CPKelco), in which the tablets comprise different drug:polymer ratios (8 mm tablet size; different tablet weights; same dose of drug).

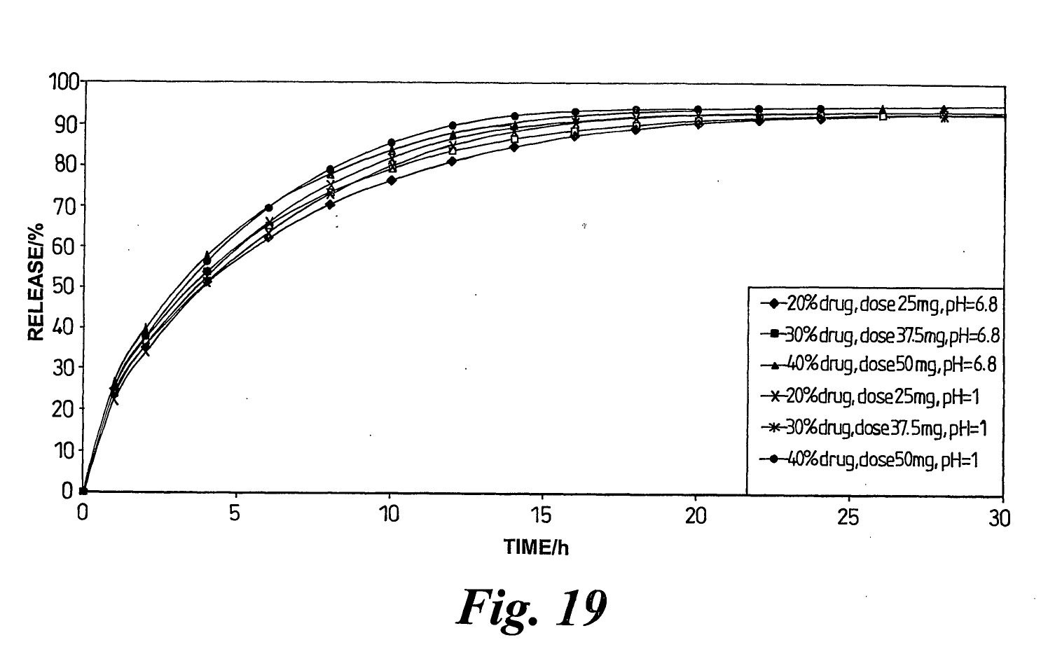

FIG. 19 shows the drug release profile at different pHs of the methanesulphonate salt of Compound D from tablets made from a specific grade of HPMC polymer (METHOCEL™ 60SH10000; Shin-Etsu), in which the tablets comprise different drug:polymer ratios (8 mm tablet size; 152 mg tablet weight; different doses of drug).

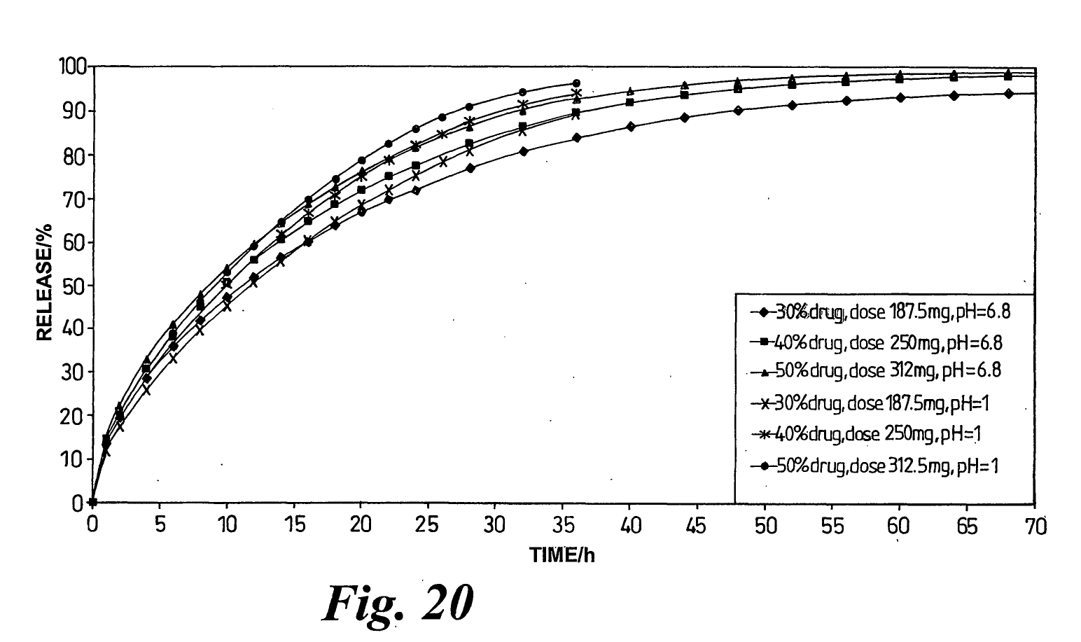

FIG. 20 shows the drug release profile at different pHs of the methanesulphonate salt of Compound D from tablets made from a specific grade of HPMC polymer (METHOCEL™ 60SH10000; Shin-Etsu), in which the tablets comprise different drug:polymer ratios (12 mm tablet size; 760 mg tablet weight; different doses of drug).

Preparation A

Preparation of Compound A and Benzenesulphonate Salt Thereof

(i) 4-[(3-Hydroxypropyl)amino]benzonitrile

Alternative 1 A mixture of 4-fluorobenzonitrile (12.0 g, 99.1 mmol) and 3-amino-1-propanol (59.6 g, 793 mmol) was stirred at 80° C. under an inert atmosphere for 3 hours before water (150 mL) was added. The mixture was allowed to cool to room temperature, and was then extracted with diethyl ether. The organic layer was separated, dried (Na₂SO₄), filtered and concentrated in vacuo to yield 17 g (97%) of the sub-title compound as an oil that crystallised upon standing.

Alternative 2 4-Fluorobenzonitrile (24.6 g, 0.203 mol, Aldrich 99%) was added to 3-amino-1-propanol (122.0 g, 1.625 mol, 8 equiv., Aldrich 99%) and the mixture heated to 80° C. for 5 hours, under nitrogen. The solution was allowed to cool to 22° C. and water (300 mL) was added. The cloudy solution was extracted twice with methylene chloride (300 mL and 200 mL) and the combined methylene chloride extracts were washed with water (300 mL; GC analysis of organic layer gave ˜1.0 area % aminopropanol remaining).

Alternative 3 To 4-fluorobenzonitrile (30.29 g, 247.7 mmol, 1.0 eq), was added 3-amino-1-propanol (150 mL, 148.8 g, 1981.5 mmol, 8.0 eq). The mixture was stirred under nitrogen at room temperature (27° C.) until all of the solid had dissolved. The solution was heated (oil bath) to 77° C. and kept at this temperature for 7 hours, before being stirred at ambient temperature overnight (14 hours). Water (365 mL) was added, and the resultant cloudy solution was extracted with dichloromethane (365 mL, then 245 mL). The combined organic layers were washed with water (365 mL). The DCM solution of the product was dried by distillation: solvent (200 mL) was removed and replaced with fresh DCM (200 mL). More solvent (250 mL) was removed to bring the total solvent volume to 365 mL.

(ii) 3-(4-Cyanoanilino)propyl 4-methylbenzenesulfonate

Alternative I A cooled (0° C.) solution of 4-[(3-hydroxypropyl)-amino]benzonitrile (from step (i) (Alternative 1) above; 17 g, 96.5 mmol) in dry MeCN (195 mL) was treated with triethylamine (9.8 g, 96.5 mmol) and then p-toluenesulfonyl chloride (20.2 g, 106 mmol). The mixture was stirred at 0° C. for 90 minutes before being concentrated in vacuo. Water (200 mL) was added to the residue, and the aqueous solution was extracted with DCM. The organic phase was dried (Na₂SO₄), filtered and concentrated in vacuo. The resulting residue was purified by crystallisation from iso-propanol to yield 24.6 g (77%) of the title compound.

Alternative II The solution of the crude 4-[(3-hydroxypropyl)amino]-benzonitrile (from step (i) (Alternative 2) above) was concentrated to a volume of 300 mL by distillation and a further 200 mL methylene chloride added and re-distilled to 300 mL (solution water by Karl-Fischer 0.07%). Triethylamine (20.55 g, 0.203 mol), followed by 4-(N,N-dinethyl-amino)pyridine (248 mg, 2.0 mmol) was added and the solution was cooled to 0° C. A solution of tosyl chloride (38.70 g, 0.203 mol) in methylene chloride (150 mL) added over ca. 30 minutes with cooling and good agitation, allowing the temperature to rise to 5° C. The reaction was stirred for 23 hours in the range 3 to 5° C. under nitrogen. (After 5 hours, triethylamine hydrochloride precipitation occurred. TLC showed very little if any further conversion of residual cyano alcohol at 20-23 hours.) Water (300 mL) was added and the layers vigorously agitated for 15 mm. The organic solution was concentrated by distillation at 35 to 40° C. to a volume of ca. 60 to 70 mL. iso-Propanol (100 mL) was added over 5 minutes. (At this stage, some granular precipitation of product occurred prior to addition of iso-propanol. Crystallization occurred rapidly upon addition of iso-propanol.) Distillation was continued using vacuum to remove the last of the methylene chloride. (A further ˜30 mL was removed and the distillate was checked by GC for the absence of methylene chloride.) The crystal slurry was cooled to 0 to 5° C. over ca. 1 hour with slow agitation and held for one hour at 0-5° C. The crystals were filtered on a medium sinter and the compacted damp filter cake carefully washed with cold (0° C.) iso-propanol (80 mL). The filter cake was dried under vacuum and a stream of nitrogen overnight. Yield: 52.6 g, 78.4 mole%; HPLC: 99.64 area %.

Microanalysis:found (theory):%C:61.60 (61.67); %H:5.41 (5.49); %N:8.44 (8.47); %S:9.71(9.70).

(iii) N,N-Bis(2-oxiranylmethyl)benzenesulphonamide

Water (2.5 L, 10 vol.) followed by epichlorohydrin (500 mL, 4 eq.) were added to benzenesulphonamide (250 g, 1 eq.). The reactants were heated to 40° C. Aqueous sodium hydroxide (130 g in 275 mL of water) was added such that the temperature of the reaction remained between 40° C. and 43° C. This took approximately 2 hours. (The rate of sodium hydroxide addition needs to be slower at the start of the addition than at the end in order to keep within the temperature range stated.) After the addition of sodium hydroxide was complete, the reaction was stirred at 40° C. for 2 hours, then at ambient temperature overnight. The excess epichlorohydrin was removed as a water azeotrope by vacuum distillation (ca. 40 mbar, internal temp 30° C.), until no more epichlorohydrin distilled. Dichloromethane (1L) was added and the mixture stirred rapidly for 15 minutes. The phases were allowed to separate (this took 10 minutes although totally clear phases are obtained after standing overnight). The phases were separated and the dichloromethane solution used in the subsequent step below.

¹HNMR (400 MHz, CDCl₃): δ2.55-2.65 (2H, m), 2.79 (2H, t,J4.4), 3.10-3.22 (4H, m), 3.58-3.73 (2H, m), 7.50-7.56 (2H, m), 7.58-7.63 (1H, m), 7.83-7.87 (2H, m).

(iv) 5-Benzyl-3,7-dihydroxy-1-phenylsulphonyl-1,5-diazacyclooctane

IMS (2.5 L, 10 vol) was added to the dichloromethane solution from step (iii) above. The solution was distilled until the internal temperature reached 70° C. Approximately 1250 mL of solvent was collected. More IMS (2.5 L, 10 vol) was added followed by benzylamine (120 mL, 0.7 eq.) in one portion (no exotherm seen), and the reaction was heated at reflux for 6 hours (no change from 2 hour sampling point). More benzylamine was added (15 mL) and the solution was heated for a further 2 hours. The IMS was distilled off (ca. 3.25 L) and toluene was added (2.5 L). More solvent was distilled (ca. 2.4 L) and then further toluene added (1 L). The head temperature was now 110° C. A further 250 mL of solvent was collected at 110° C. Theoretically, this left the product in ca. 2.4 L of toluene at 110° C. This solution was used in the next step.

¹H NMR (400 MHz, CDCl₃): δ7.83-7.80 (4H, m, ArH), 7.63-7.51 (6H, m, ArH), 7.30-7.21 (10H, ArH), 3.89-3.80 (4H, m, CH(a) +CH(b)), 3.73 (2H, s, CH₂Ph(a)), 3.70 (2H, s, CH₂Ph(b)), 3.59 (2H, dd, CHHNSO2Ar(a)), 3.54 (2H, dd, CHHNSO₂Ar(b)), 3.40 (2H, dd, CHHNSO₂Ar(b)), 3.23 (2H, dd, CHHNSO₂Ar(a)), 3.09-2.97 (4H, m, CHHNBn(a)+CHHNBn(b)), 2.83 (2H, dd, CHHNBn(b)), 2.71 (2H, dd, CHHNBn(a)) (Data taken from purified material comprising a 1:1 mixture of trans- (a), and cis-diol (b))

(v) 3-Benzyl-7-(phenylsulphonyl)-9-oxa-3,7-diazabicyclo[3.3.1 ]nonane

The toluene solution from the previous step (iv) above was cooled to 50° C. Anhydrous methanesulphonic acid (0.2 L) was added. This caused a temperature rise from 50° C. to 64° C. After 10 minutes, methanesulphonic acid was added (1 L) and the reaction heated to 110° C. for 5 hours. Toluene was then distilled from the reaction; 1.23 L was collected. (Note that the internal temperature should not be allowed higher than 110° C. at any stage otherwise the yield will be decreased.) The reaction was then cooled to 50° C. and a vacuum applied to remove the rest of the toluene. Heating to 110° C. and 650 mbar allowed a further 0.53 L to be removed. (If the toluene can be removed at a lower temperature and pressure then that is beneficial.) The reaction was then left to cool to 30° C. and deionised water (250 mL) was added. This caused the temperature to rise from 30° C. to 45° C. More water (2.15 L) was added over a total time of 30 minutes such that the temperature was less than 54° C. The solution was cooled to 30° C. and then dichloromethane (2 L) was added. With external cooling and rapid stirring, the reaction mixture was basified by adding aqueous sodium hydroxide (10 M, 2 L) at a rate that kept the internal temperature below 38° C. This took 80 minutes. The stirring was stopped and the phases separated in 3 minutes. The layers were partitioned. IMS (2 L) was added to the dichloromethane solution and distillation started. Solvent (2.44 L) was collected until the head temperature reached 70° C. Theoretically, this left the product in 1.56 L of IMS. The solution was then allowed to cool to ambient temperature overnight with slow stirring. The solid product that precipitated was filtered and washed with IMS (0.5 L) to give a fawn-coloured product that, on drying at 50° C, in vacuum, gave 50.8 g (8.9% over 3 steps). 20.0 g of this product was dissolved in acetonitrile (100 mL) at reflux to give a pale yellow solution. After cooling to ambient temperature, the crystals that formed were collected by filtration and washed with acetonitrile (100 mL). The product was dried in vacuo at 40° C. for 1 hour to give 17.5 g (87%) of sub-title compound. ¹H NMR (400 MHz, CDCl₃): δ7.18-7.23 (1OH, m), 3.86-3.84 (2H, m), 3.67 (2H, d), 3.46 (2H, s), 2.91 (2H, d), 2.85 (2H, dd), 2.56 (2H, dd)

(vi) 3-Benzyl-9-oxa-3,7-diazabicyclo[3.3.1]nonane×2 HCl

Concentrated hydrobromic acid (1.2 L, 3 rel. vol.) was added to solid 3-benzyl-7-(phenylsulphonyl)-9-oxa-3,7-diazabicyclo[3.3.1]nonane (400 g, see step (v) above) and the mixture was heated to reflux under a nitrogen atmosphere. The solid dissolved in the acid at 95° C. After heating the reaction for 8 hours, HPLC analysis showed that the reaction was complete. The contents were cooled to room temperature. Toluene (1.2 L, 3 rel. vol.) was added and the mixture stirred vigorously for 15 minutes. Stirring was stopped and the phases were partitioned. The toluene phase was discarded along with a small amount of interfacial material. The acidic phase was returned to the original reaction vessel and sodium hydroxide (10 M, 1.4 L, 3.5 rel. vol.) was added in one portion. The internal temperature rose from 30° C. to 80° C. The pH was checked to ensure it was >14. Toluene (1.6 L, 4 rel. vol.) was added and the temperature fell from 80° C. to 60° C. After vigorous stirring for 30 minutes, the phases were partitioned. The aqueous layer was discarded along with a small amount of interfacial material. The toluene phase was returned to the original reaction vessel, and 2-propanol (4 L, 10 rel. vol.) was added. The temperature was adjusted to between 40° C. and 45° C. Concentrated hydrochloric acid (200 mL) was added over 45 minutes such that the temperature remained at between 40° C. and 45° C. A white precipitate formed. The mixture was stirred for 30 minutes and then cooled to 7° C. The product was collected by filtration, washed with 2-propanol (0.8 L, 2 rel vol.), dried by suction and then further dried in a vacuum oven at 40° C. Yield=297 g (91%).

¹H NMR (CD₃OD+4 drops D₂O): δ2.70 (br d, 2H), 3.09 (d, 2H), 3.47 (br S, 4H), 3.60 (s, 2H), 4.12 (br s, 2H), 7.30-7.45 (m, 5H). API MS: m/z=219 [C₁₃H₁₈N₂O+H]⁺.

(vii) 3,3-Dimethyl-l-[9-oxa-7-(phenylmethyl)-3,7-diazabicyclo[3.3.1]non-3-yl ]-2-butanone

Water (500 mL, 5 vol.) followed by 1-chloropinacolone (45.8 mL, 1 eq.) were added to sodium bicarbonate (114.2 g, 4 eq.). A solution of 3-benzyl-9-oxa-3,7-diazabicyclo[3.3.1]nonane×2 HCl (100.0 g; see step (vi) above) in water (300 mL, 3 vol.) was added slowly, so that the evolution of carbon dioxide was controlled (20 mins.). The reaction mixture was heated at 65 to 70° C. for 4 hours. After cooling to ambient temperature, dichloromethane (400 mL, 4 vol.) was added and, after stirring for 15 minutes, the phases were separated. The aqueous phase was washed with dichloromethane (400 mL, 4 vol.) and the organic extracts combined. The solution was distilled and solvent collected (550 mL). Ethanol (1 L) was added and the distillation continued. Further solvent was collected (600 mL). Ethanol (1 L) was added and the distillation continued. Further solvent was collected (500 mL) (the head temperature was now 77° C.). This solution (theoretically containing 1150 mL of ethanol) was used directly in the next step.

¹H NMR (400 MHz, CDCl₃): δ1.21 (9H, s), 2.01-2.59 (2H, m), 2.61-2.65 (2H, m), 2.87-2.98 (4H, m), 3.30 (2H, s), 3.52 (2H, s), 3.87 (2H, br s), 7.26 (2H, d, J7.6), 7.33 (1H, dd, J7.6, 7.6), 7.47 (2H, d, J7.6).

(viii) 3,3-Dimethyl-1-(9-oxa-3,7-diazabicyclo[3.3.1]non-3 -yl)-2-butanone

Palladium on charcoal (44 g, 0.4 wt. eq. of 61% wet catalyst, Johnson Matthey Type 440L) was added to the ethanol solution from the previous step (vii) above. The mixture was hydrogenated at 4 bar. The reaction was considered complete after 5 hours. The catalyst was removed by filtration and washed with ethanol (200 mL). The combined ethanol filtrates were/may be used in step (ix) below. Solution assay gave 61.8 g of title product in ethanol (theoretically 1.35 L; measured 1.65 L). A portion of the product was isolated and purified. Analysis was performed on the purified product.

¹H NMR (300 MHz, CDCl₃): δ1.17 (9H, s), 2.69 (2H, dt, J 11.4, 2.4), 2.93 (2H, d, J 10.8), 3.02 (2H, d, J 13.8), 3.26 (2H, s), 3.32 (2H, dt, J 14.1), 3.61 (2H, br s).

This reaction may also be performed using a lower weight ratio of catalyst to benzylated starting material. This may be achieved in several different ways, for example by using different catalysts (such as Pd/C with a metal loading different from that in the Type 440L catalyst employed above, or Rh/C) and/or by improving the mass transfer properties of the reaction mixture (the skilled person will appreciate that improved mass transfer may be obtained, for example, by performing the hydrogenation on a scale larger than that described in the above reaction). Using such techniques, the weight ratio of catalyst to starting material may be reduced below 4:10 (e.g. between 4:10 and 1:20.).

(ix) Compound A, benzenesulphonic acid salt monohydrate

Method 1

Potassium carbonate (56.6 g, 1.5 equiv) and 3-(4-cyanoanilino)propyl-4-methylbenzenesulphonate (see step (ii) above, 90.3 g, 1 equiv) were added to an ethanol solution of 3,3-dimethyl-1-(9-oxa-3,7-diazabicyclo[3.3.1]non-3-yl)-2-butanone (see step (viii) above; 61.8 g from assay in 1.65 L). The reaction was heated at 80° C. for 4 hours. An assay showed some reactant remained (8.3 g), so more 3-(4-cyanoanilino)propyl-4-methylbenzenesulphonate (12.2 g) was added, and the resultant was heated at 80° C. for 4 hours. Solvent (1.35 L) was distilled, then iso-propyl acetate (2.5 L) added. Solvent (2.51 L) was removed. iso-Propyl acetate (2.5 L) was added. Solvent (0.725 L) was removed. The internal temperature was now at 88° C. Solvent (0.825 L) was removed, leaving the product as an iso-propyl acetate solution (theoretically in 2.04 L). After cooling to 34° C., water (0.5 L) was added. There was a black suspension, possibly of Pd, in the mixture. The pH of the aqueous phase was 11. Sodium hydroxide (1 M, 0.31 L) was added, so that the temperature was less than 25° C., and the mixture was stirred vigourously for 5 minutes. The pH of the aqueous phase was 12. The phases were separated and the aqueous phase discarded. More water (0.5 L) was added, and the phases were separated. The aqueous phase was discarded. The remaining ester solution was filtered to remove suspended particles, and the filtrate was then made up to exactly 2 L. The solution was then split into 2×1 L portions.

(In order to avoid producing sub-title product comprising a high palladium content, the following treatment may be performed: Deloxan® resin (12.5 g, 25 wt %) was added to the solution of the free base (1 L), and the mixture heated at reflux with vigorous stirring for 5 hours. The solution was then cooled to room temperature, and was stirred for 2 days. The resin was removed by filtration.)

An assay was performed to calculate the required amount of benzenesulphonic acid, to make the benzenesulphonate salt.

A solution of benzenesulphonic acid (20.04 g, 1 eq., assuming acid was pure monohydrate) in isopropyl acetate (200 mL) was added over 5 minutes (better to add slower if possible) with vigorous stirring to the solution of the free base (1 L) and a pale yellow precipitate formed. The temperature rose from 18° C. to 22° C. After 10 minutes, the mixture was cooled to 10° C. and the product collected by filtration. The product was washed with iso-propyl acetate (250 mL), sucked dry on the filter then dried under vacuum at 40° C. for 2 days to give 59.0 g (61% from 3-benzyl-9-oxa-3,7-diazabicyclo[3.3.1]nonane×2HCl).

(The crude benzenesulphonate salt was alternatively prepared by the addition of a 70% (w/w) aqueous solution of benzenesulphonic acid to an ethanolic solution of the free base.)

The crude sub-title product is isolated as a monohydrate.

Ethanol (500 mL) and water (250 mL) were added to crude sub-title compound (50.0 g). The solution was heated to 75° C. Material was all dissolved at 55° C. The solution was held at 75° C. for 5 minutes, then cooled to 5° C. over 1 hour. Precipitation started at 18° C. The cold solution was filtered and the filtrate washed with ethanol:water (2:1; 150 mL), sucked dry on the filter, and then dried in vacuo at 40° C. to give pure sub-title product (41.2 g, 82%).

(This recrystallisation may be carried out with greater volumes of solvent if necessary to fit the reaction vessels e.g. EtOH: water 2:1, 45 vol. (gave 62% recovery) EtOH: water 6:1, 35 vol. (gave 70% recovery).)

The sub-title product was isolated as the monohydrate following the rescrystallisation (as determined by single crystal X-ray diffraction).

Method 2

(a) 3-(4-Cyanoanilino)propyl benzenesulfonate

To the solution of 4-[(3-hydroxypropyl)amino]benzonitrile (from step (i) Alternative 3 above, assumed 43.65 g, 247.7 mmol, 1.0 eq) in dichloromethane (360 mL total solution volume) was added, sequentially, triethylamine (52 mL, 37.60 g, 371.55 mmol, 1.5 eq) and trimethylamine hydrochloride (11.89 g, 123.85 mmol, 0.5 eq) in one portion. The yellow solution was cooled to −20° C. (using an acetone/dry ice bath or a cold plate), and treated with a solution of benzenesulfonyl chloride (32 mL, 43.74 g, 247.7 mmol, 1.0 eq) in dichloromethane (220 mL, 5 vols with respect to the cyanoalcohol) via a pressure equalising dropping funnel. The solution was added portionwise such that the internal temperature did not exceed −14° C. The addition took 25 minutes to complete. The mixture was then stirred for minutes at between −15 and −10° C. Water (365 mL) was added and the temperature rose to 10° C. The mixture was cooled back to 0° C. and stirred vigorously for 15 minutes. The organic layer (volume 570 mL) was collected and distilled at atmospheric pressure to remove DCM (450 mL, pot temperature 40-42° C., still-head temperature 38-39° C). Ethanol (250 mL) was added, and the solution was allowed to cool to below 30° C. before turning on the vacuum. More solvent was removed (40 mL was collected, pressure 5.2 kPa (52 mbar), pot and still-head temperatures were 21-23° C.), and the product gradually came out of solution. The distillation was stopped at this point, and more ethanol (50 mL) was added. The mixture was warmed (hot water bath at 50° C.) to 40° C. to dissolve all the solid, and water (90 mL) was added slowly via a dropping funnel. The solution was stirred slowly at room temperature (20° C.) overnight (15 hours), by which time some product had crystallised out. The mixture was cooled to −5° C. (ice/methanol bath) and stirred at this temperature for 20 minutes before collecting the pale yellow solid by filtration. The solid was washed with an ethanol/water mixture (42 mL EtOH, 8 mL H₂O ),, and suction dried for 30 minutes before drying to constant weight in the vacuum oven (40° C., 72 hours). The mass of crude product obtained was 47.42 g (149.9 mmole, 60%). Ethanol (160 mL, 8 vols) was added to the crude product (20.00 g, 63.22 mmol, 1.0 eq). The mixture was stirred under nitrogen and warmed to 40° C. using a hot water bath. On reaching this temperature, all of the solid had dissolved to give a clear, yellow solution. Water (60 mL, 3 vols) was added dropwise over a period of 10 minutes, whilst the internal temperature was maintained in the range 38-41° C. The water bath was removed, and the solution was allowed to cool to 25° C. over 40 minutes, by which time crystallisation had begun. The mixture was cooled to −5° C. over 10 minutes, then held at this temperature for a further 10 minutes. The pale yellow solid was collected by filtration, suction dried for 10 minutes, then dried to constant weight in a vacuum oven (40° C., 15 hours). The mass of sub-title compound obtained was 18.51 g (58.51 mmol, 93% (from the crude product)).

(b) Compound A, benzenesulphonic acid salt monohydrate

To an ethanol solution (total volume 770 mL, approx. 20 vols with respect to the amine) of 3,3-dimethyl-1-(9-oxa-3,7-diazabicyclo[3.3.1]non-3-yl)-2-butanone (assumed 34.97 g (verified by assay), 154.5 mmol, 1.0 eq; see step (viii) above) was added 3-(4-cyanoanilino)propyl benzenesulfonate (49.05 g, 154.52 mmol, 1.0 eq; see step (a) above) in one portion. The resultant mixture was heated at 74° C. for 6 hours, then stirred at room temperature (20° C.) for 65 hours (over the weekend; the skilled person will appreciate that the reaction will also succeed without this prolonged stirring at room temperature). Ethanol (370 mL) was removed, and water (200 mL) was added (this gave a 2:1 EtOH:H₂O mixture, total volume 600 mL). Upon adding the water, the pot temperature fell from 80° C. to 61° C. The solution was re-heated to 70° C., then allowed to cool naturally to ambient temperature overnight (19 hours), whilst stirring slowly. A solid was observed at this stage. The mixture was cooled to 0° C. and then stirred at this temperature for 15 minutes before collecting the off-white solid by filtration. The solid was washed with a cold 2:1 mixture of ethanol:water (150 mL), suction dried for 1.25 hours, then oven-dried (40° C., 20 hours). The mass of crude product obtained was 57.91 g (103.3 mmol, 60%).

The crude product was found to be 98.47% pure (as determined by HPLC analysis), and was recrystallised (using the procedure detailed below) to give the sub-title compound in a purity of 99.75% (84% recovery).

Recrystallisation procedure:

Ethanol (562 mL) and water (281 mL) were added to the crude product obtained above (56.2 g). The solution was heated to 75° C. All material dissolved at 55° C. The solution was held at 75° C. for 5 minutes, before being cooled to 5° C. over 1.5 hours. Precipitation started at 35° C. The cold solution was filtered and the collected precipitate was washed with ethanol: water (2:1, 168 mL). The solid material was sucked dry on the filter, before being dried in vacuo at 40° C. to give product (47.1 g, 84%).

(x) Compound A (free base)

Method I

Crude benzenesulphonate salt (50.0 g, 1.0 equiv, from step (ix) above; Method 1) was added to aqueous sodium hydroxide (1M, 500 mL) washing in with dichloromethane (1.0 L, 20 vol). The combined mixture was stirred for 15 minutes. The layers were then separated and a small amount of interfacial material was left with the upper aqueous layer. Ethanol (500 mL, 10 vol) was added to the dichloromethane solution and then solvent was removed by distillation (1.25 L). The still head temperature was now at 78° C. The solution was allowed to cool to below reflux and ethanol (250 mL, 5 vol.) was added. Solvent was removed (250 mL). This warm solution was diluted with ethanol to 890 mL, 17.8 vol. (25 vol. assuming 100% conversion to free base). After heating to reflux the solution was cooled slowly. At 5° C. a seed of title compound was added. Crystallisation began and the mixture was stirred. at 5° C. for 30 minutes. The product was collected by filtration and washed with ethanol (2×50 mL, 2×1 vol.). The product was then dried in a vacuum oven at 40° C for 60 hours to give an off-white powder (26.3 g; 74%). ¹H NMR (400 MHz, CDCl₃): δ7.86-7.82 (2H, m), 7.39-7.32 (3H. m), 7.30-7.26 (2H, m), 6.47 (2H, m), 4.11-4.07 (4H, m), 3.70 (2H, s), 3.36-3.33 (4H, m), 3.26 (2H, t), 3.12 (2H, d), 2.90 (2H, d), 2.28-2.21 (2H, m), 1.06 (9H, s). ¹³C NMR (CDCl₃): δ24.07, 26.38, 41.52, 43.52, 56.17, 56.47, 63.17, 68.46, 96.61, 111.64, 121.03, 133.43. MS (ES): m/z=385.1 (M+H)⁺

Method II

A mixture of 4-{[3-(9-oxa-3,7-diazabicyclo[3.3.1]non-3-yl)propyl]amino}-benzonitrile (see Preparation B(I)(vi) below; 5.73 g, 0.02 mol), K₂CO₃ (11.05 g, 0.08 mol) in MeCN (300 mL) was treated with 1-chloropinacolone (4.44 g, 0.032 mol). The mixture was stirred at 50° C. overnight before DCM was added and the mixture filtered. The filter cake was then washed with a mixture of DCM and MeCN before the solvent was evaporated from the filtrate. The resulting residue was purified by chromatography on silica, eluting with a gradient of ethyl acetate:methanol:ammoniacal methanol (95:5:0 to 95:0:5), to give the title compound (5.8 g, 73.9%).

Preparation B(I)

Preparation of Compound B (Method I)

(i) tert-Butyl 2-bromoethylcarbamate

Sodium bicarbonate (6.15 g, 0.073 mol) and di-t-butyl dicarbonate (11.18 g, 0.051 mol) were dissolved in a mixture of H₂O (50 mL) and dichloromethane (150 mL), then cooled to 0° C. 2-Bromoethylamine hydrobromide (10.0 g, 0.049 mol) was added slowly as a solid, and the reaction was stirred overnight at 25° C. The dichloromethane layer was separated, washed with H₂O (200 mL) and washed with a solution of potassium hydrogensulphate (150 mL, pH=3.5). The organic layer was dried (Na₂SO₄) and concentrated in vacuo. The crude oil was chromatographed on silica gel, eluting with dichloromethane to afford 7.87 g (72%) of the sub-title compound as a clear, colorless oil. ¹H NMR (300 MHz, CDCl₃) δ4.98 (bs, 1H), 3.45-3.57 (m, 4H), 1.47 (s, 9H) API-MS: (M+1-C₅H₈O₂) 126 m/z

(ii) 3-Benzyl-9-oxa-3,7-diazabicyclo[3.3.1]nonane×2 HCl

This is an alternative preparation to that described in Preparation A(vi) above. A 3L, three-necked flask was equipped with a magnetic stirrer, a thermometer and a reflux condenser. Aqueous hydrobromic acid (48%, 0.76 L, 4.51 mol) was added to solid 3-benzyl-7-(phenylsulphonyl)-9-oxa-3,7-diazabicyclo[3.3.1]nonane (190 g, 0.53 mol, see Preparation A(v) above) and the mixture was heated to reflux under nitrogen. The solid dissolved at 90° C. After heating the mixture for 12 hours, GC analysis showed that the reaction was complete. The contents were cooled to room temperature. Toluene (0.6L) was added and the mixture was stirred for a few minutes. The phases were separated. The aqueous phase was returned to the original reaction vessel and aqueous sodium hydroxide (10M, 0.85 L, 8.5 mol) was added in one portion. The internal temperature rose to 80° C. and the mixture was strongly basic. Toluene (0.8 L) was added when the internal temperature dropped to 55° C. After stirring vigorously for 30 minutes, the toluene phase was separated and returned to the original reaction vessel. 2-Propanol (1.9 L) was added and the internal temperature was adjusted to between 40° C. and 50° C. Concentrated hydrochloric acid was added (until acidic) at such a rate to maintain the temperature between 40° C. and −50° C. A white precipitate formed. The mixture was stirred for 30 minutes and then cooled to 7° C. The white powder was collected by filtration, washed with 2-propanol (0.4 L), dried by pulling air through the sample for ten minutes, and then further dried in a vacuum oven at 40° C. Yield: 130 g (84%).

(iii) tert-Butyl 7-benzyl-9-oxa-3,7-diazabicyclo[3.3.1]nonane-3-carboxylate hydrochloride

A 5L, three-necked flask was equipped with an overhead stirrer, a thermometer and a nitrogen bubbler. Water (1.4 L), dichloromethane (1.4 L), sodium bicarbonate (150 g, 1.79 mol) and 3-benzyl-9-oxa-3,7-diazabicyclo [3.3.1]nonane×2 HCl (130 g, 0447 mol, from step (ii) above) were all charged in order. The mixture was stirred rapidly for ten minutes and then di-tert-butyl dicarbonate (0.113 L, 0.491 mol) was added slowly. The mixture was stirred rapidly for three hours at room temperature. The organic layer was separated, dried with magnesium sulfate, filtered and concentrated to afford 160 g of an off-white solid. The off-white solid was charged into a 3L, three-necked flask equipped with an overhead stirrer, a thermometer and an addition funnel. Ethyl acetate (0.6 L) was charged and the clear solution was cooled to −10° C. A solution of HCl in dioxane (4 M) was added dropwise until the pH was less than 4. The hydrochloride salt precipitated and the mixture was stirred for an additional hour. The product was collected by filtration, washed with ethyl acetate (0.1 L), and dried overnight in a vacuum oven. The white crystalline product weighed 146 g (92% yield).

(iv) tert-Butyl 9-oxa-3,7-diazabicyclo[3.3.1 ]nonane-3-carboxylate hydrochloride

Hydrochloride salt from step (iii) above (146 g, 0.411 mol) and 20% Pd(OH)₂—C (7.5 g) were charged to a Parr hydrogenator bottle. Methanol (0.5 L) was added and the bottle was shaken vigorously under an atmosphere of hydrogen at 3.5 bar. The reaction was monitored by GC analysis and was found to be complete after one hour. The catalyst was filtered and the filtrate was concentrated to afford an off-white crystalline product. The crude product was dissolved in hot acetonitrile (1.2 L), and then filtered while hot. The filtrate was diluted with ethyl acetate (1.2 L). The clear solution was allowed to stand overnight at room temperature. The first crop of crystals was collected and dried under vacuum to afford 52 g of sub-title compound as a white solid. The filtrate was concentrated to near dryness, then dissolved in hot acetonitrile (0.4 L), and diluted with ethyl acetate (0.4 L). A second crop of crystals (38 g) was obtained after cooling the solution to 10° C. Both crops were found to be comparable by GC analysis and ¹H NMR analyses. Combined yield: 90 g (83%).

(v) tert-Butyl 7-[3-(4-cyanoanilino)propyl]-9-oxa-3,7-diazabicyclo[3.3.1]-nonane-3-carboxylate

The hydrochloride salt of tert-butyl 9-oxa-3,7-diazabicyclo[3.3.1]nonane-3-carboxylate (see step (iv) above; 1.1 g, 4.15 mmol) was mixed with MeCN (46 mL), water (2.5 mL) and K₂CO₃ (3.5 g, 25 mmol). The mixture was stirred for 4 h before CHCl₃ was added and the mixture was filtered through Celite®. The filtrate was concentrated in vacuo to give 0.933 g of the free base. This was then mixed with 3-(4-cyanoanilino)propyl 4-methylbenzenesulphonate (see Preparation A(ii) above; 2.1 g, 6.2 mmol) and K₂CO₃ (0.86 g, 6.2 mmol) in MeCN (18 mL). The resulting mixture was stirred overnight at 60° C. before being concentrated in vacuo. The residue was treated with DCM (250 mL) and 1 M NaOH (50 mL). The layers were separated and the DCM layer washed twice with aqueous NaHCO₃, before being dried (Na₂SO₄) and concentrated in vacuo. The product was purified by flash chromatography, eluting with a gradient of toluene:ethyl acetate:triethylamine (2:1:0 to 1000:1000:1), to give 1.47 g (91%) of the sub-title compound.

(vi) 4- {[3-(9-Oxa-3,7-diazabicyclo[3.3.1 ]non-3-yl)propyl]amino}benzonitrile

The sub-title compound was obtained in 96% yield using an analogous procedure to those described in Preparations C(v) and D(iii) below, using tert-butyl 7-[3-(4-cyanoanilino)propyl]-9-oxa-3,7-diazabicyclo[3.3.1]-nonane-3-carboxylate (from step (v) above).

(vii) Compound B

To a solution of tert-butyl 2-bromoethylcarbainate (4.21 g, 0.019 mol; see step (i) above) in DMF (65 mL) was added 4-{[3-(9-oxa-3,7-diazabicyclo[3.3.1]non-3-yl)propyl ]amino}benzonitrile (see step (vi) above, 4.48 g, 0.016 mol) and triethylamine (3.27 mL, 0.024 mol). The mixture was stirred overnight at 35° C. and then concentrated in vacuo. The residue was dissolved in dichloromethane (80 mL) and washed with saturated sodium chloride. The aqueous layer was extracted with dichloromethane (1 ×150 mL). The combined organic extracts were dried (Na₂SO₄) and concentrated in vacuo. The crude red-brown oil was chromatographed (×2) on silica gel eluting with chloroform:methanol:conc. NH₄OH (9:1:0.02) to afford 3.75 g (56%) of the title compound. ¹H NMR (300 MHz, CD₃OD) δ7.37-7.40 (d, J=8.8 Hz, 2H), 6.64-6.67 (d, J=8.8 Hz, 2H), 3.94 (bs, 2H), 3.21-3.31 (m, 4H), 3.01 (bs, 4H), 2.47-2.59 (m, 8H), 1.90 (bs, 2H), 1.39 (s, 9H) ¹³C NMR (75 MHz, CD₃OD) δ158.5, 134.7, 121.9, 113.2, 97.7, 80.3, 69.2, 58.8, 58.1, 57.5, 57.3, 41.9, 38.3, 28.9, 26.2. API-MS: (M+1)=430 m/z

Preparation B(II)

Preparation of Compound B (Method II)

(i) [2-(7-Benzyl-9-oxa-3,7-diazabicyclo[3.3.1]non-3-yl)ethyl]carbamic acid tert-butyl ester

Alternative 1

(a) 2-(tert-Butyloxycarbonylamino)ethyl tosylate

A solution of p-toluenesulfonyl chloride (28.40 g, 148 mmol) in dichloromethane (100 mL) was added dropwise over 30 minutes at 0° C. to a mixture of tert-butyl N-(2-hydroxyethyl)carbamate (20 g, 120 mmol), triethylamine (18.80 g, 186 mmol) and trimethylammonium chloride (1.18 g, 12.4 mmol) in dichloromethane (120 mL). The mixture was stirred at 0° C. for 1 hour then filtered, washing with dichloromethane (100 mL).

The filtrate was washed with 10% citric acid (3×100 mL) and brine (100 mL). The organic layer was dried with magnesium sulfate and then filtered. The filtrate was concentrated under reduced pressure to give an oil. The oil was dissolved in ethyl acetate (40 mL) and then iso-hexane (160 mL) was added slowly. The resultant -slurry was stirred at room temperature for 17 hours and then filtered. The collected solid was washed with iso-hexane (240 mL) to yield the sub-title compound as a colourless powder (25 g, 64%). m.p. 64-66° C. ¹H-NMR (300 MHz, CDCl₃,) δ1.40 (9H, s), 2.45 (3H, s), 3.38 (2H, q), 4.07 (2H, t), 4.83 (1H, bs) 7.34 (2H, d), 7.87 (2H, d). MS:m/z=216 (MH⁺(316)-Boc).

(b) [2-(7-Benzyl-9-oxa-3,7-diazabicyclo[3.3.1]non-3-yl)ethyl]carbamic acid tert-butyl ester

A solution of 3-benzyl-9-oxa-3,7-diazabicyclo[3.3.1]nonane dihydrochloride (see Preparation A(vi) above; 10 g, 34 mmol) in water (25 mL) was added slowly to a solution of sodium bicarbonate (10 g, 119 mmol) in water 10 mL). More water (5 mL) was added and the mixture was stirred at room temperature for 10 minutes. A solution of 2-(tert-butyloxycarbonylamino)ethyl tosylate (see step (a) above; 11.92 g, 37 mmol) in toluene (40 mL) was added. This mixture was then heated at 65-70° C. for 7 hours before stirring at room temperature overnight. The reaction was reheated to 50° C. and the phases were separated. The aqueous layer was extracted with toluene (40 mL) at 50° C. The combined organic layers were washed with saturated sodium bicarbonate (25 mL). The solvents were evaporated under reduced pressure to yield a mixture of oil and solid (13 g, >100%). Ethyl acetate (50 mL) and citric acid (10%, 25 mL) were added to a portion of the oily solid (5 g, 138 mmol). The aqueous layer was separated and the organic layer washed again with citric acid (10%, 20 mL). The aqueous layers were combined and treated with solid sodium bicarbonate until neutral. The aqueous phase was extracted with ethyl acetate (2×50 mL), dried overmagnesium sulfate and filtered. The filtrate was evaporated to dryness under reduced pressure to give the sub-title compound as a colourless semi-solid, which solidified fully when stored in the refrigerator (4.68 g, 93%). m.p. 58-60° C. ¹H-NMR (300 MHz, CDCl₃) δ1.46 (9H, s), 2.38-2.57 (4H, m), 2.6-2.68 (2H, m) 2.75-2.85 (4H, m), 3.22 (2H, q), 3.26 (2H, s), 3.83 (2H, bs), 6.17 (1H, bs) 7.2-7.4 (5H, m). MS: m/z=362 (MH⁺).

Alternative 2

(a) 3-(7-Benzyl-9-oxa-3,7-diazabicyclo[3.3.1]non-3-yl)ethyl]propionamide

Triethylamine (3.60 g, 35.7 mmol) was added slowly to a solution of 3-benzyl-9-oxa-3,7-diazabicyclo[3.3.1]nonane dihydrochloride (see Preparation A(vi) above; 5 g, 17 mmol) in ethanol (50 mL). Acrylamide (1.34 g, 18 mmol) was added to this mixture, which was then heated at reflux for 7 hours. The reaction mixture was then concentrated under reduced pressure. Water (50 mL) and sodium hydroxide (1 M, 150 mL) were added to the residue and the mixture extracted with ethyl acetate (2×200 mL). The combined organic extracts were dried over magnesium sulfate, filtered and concentrated under reduced pressure to give a colourless solid. This was recrystallised from ethyl acetate (50 mL) to give the sub-title compound (3.80 g, 76%). m.p. 157-159° C. ¹H-NMR (300 MHz, CDCl₃) δ2.39 (2H, t), 2.42-2.61 (6H, m), 2.82-2.95 (4H, m), 3.39 (2H, s), 3.91 (2H, bs), 5.07 (1H, bs), 7.18-7.21 (2H, m), 7.25-7.39 (3H, m), 9.5 (1H, bs). MS: m/z=290 (MH⁺).

(b) [2-(7-Benzyl-9-oxa-3,7-diazabicyclo[3.3.1]non-3-yl)ethyl]carbamic acid tert-butyl ester

N-Bromosuccinimide (6.0 g, 33 mmol) was added in portions over 1 minute to a solution of 3-(7-benzyl-9-oxa-3,7-diazabicyclo[3.3.1]non-3-yl)ethyl]-propionamide (see step (a) above; 5 g, 12 mmol) in potassium tert-butoxide in tert-butanol (1 M, 81 mL) and tert-butanol (20 mL). The mixture was then heated at 60-65° C. for 30 minutes. The reaction was allowed to come to room temperature and then water (100 mL) was added. The mixture was extracted with ethyl acetate (2×50 mL). The combined organic extracts were washed with brine (50 mL), dried over magnesium sulfate, filtered (washing the filter cake with ethyl acetate (50 mL)) and then the filtrate concentrated under reduced pressure to give the sub-title compound as a brown oil (6.5 g, >100%). ¹H-NMR (300 MHz, CDCl₃) δ1.46 (9H, s), 2.4-2.58 (4H, m), 2.58-2.7 (2H, m) 2.75-2.91 (4H, m), 3.22 (2H, q), 3.28 (2H, s), 3.83 (2H, bs), 6.19 (1H, bs) 7.2-7.42 (5H, m). MS: m/z=316 (MH⁺).

Alternative 3

(a) 3-Benzyl-9-oxa-3,7-diazabicyclo[3.3.1]nonane

All volumes and equivalents are measured with respect to the amount of 3-benzyl-9-oxa-3,7-diazabicyclo[3.3.1]nonane dihydrochloride (see Preparation A(vi) above) used. Toluene (420 mL, 7 vols) and aqueous sodium hydroxide solution (2M, 420 mL, 7 vols, 4.0 eq) were added to 3-benzyl-9-oxa-3,7-diazabicyclo[3.3.1]nonane dihydrochloride (60.07 g, 206.03 mmole, 1.0 eq., see Preparation A(vi) above). The mixture was stirred under nitrogen, heated to 60° C. and held at this temperature for 30 minutes by which time two clear layers had formed. The lower, aqueous layer was removed, and the toluene solution of sub-title compound (free base) was azeodried at atmospheric pressure (total volume of solvent removed=430 mL; total volume of toluene added=430 mL), then concentrated to a volume of 240 mL (4 vols). Karl Fischer analysis at this stage showed 0.06% water in the solution. The dried solution of sub-title compound (theoretically 44.98 g, 206.03 mmole, 1.0 eq) was used as such in a subsequent step.

(b) 2-(tert-Butyloxycarbonylamino)ethyl 2,4,6-trimethylbenzenesulfonate

Triethylamine (65 mL, 465.3 mmole, 1.5 eq) was added in one portion to a solution of tert-butyl N-(2-hydroxyethyl)carbamate (50.11 g, 310.2 mmole, 1.0 eq.) in dichloromethane (250 mL, 5 vols). The solution was cooled to −10° C. and trimethylamine hydrochloride (14.84 g, 155.1 mmole, 0.5 eq.) was added in one portion. The resultant mixture was cooled further to −15° C., stirred for 5 minutes, then treated with a solution of mesitylenesulfonyl chloride (74.74 g, 341.2 mmole, 1.1 eq) in dichloromethane (250 mL, 5 vols), over 28 minutes such that the internal temperature remained below −10° C. Once the addition was complete a precipitate had formed and the mixture was stirred at −10° C. for a further 30 minutes. Water (400 mL, 8 vols) was added and all of the precipitate dissolved. The mixture was stirred rapidly for 5 minutes, and then the two layers were separated. A solvent swap from dichloromethane to iso-propanol was carried out by distillation at reduced pressure. Solvent was removed (450 mL) and replaced with iso-propanol (450 mL) (initial pressure was 450 mbar, b.p. 24° C.; final pressure was 110 mbar, b.p. 36° C.). At the end of the distillation, solvent (150 mL) was removed to bring the volume down to 350 mL (7 vols with respect to the amount of tert-butyl N-(2-hydroxyethyl)carbamate used). The solution was cooled to 25° C., then water (175 mL) was added slowly with stirring, causing the solution gradually to turn cloudy. No solid had precipitated at this stage. More water (125 mL) was added, and a solid precipitate started to form after about 75 mL had been added. The internal temperature rose from 25° C. to 31 ° C. The mixture was stirred slowly and cooled to 7° C. The solid was collected by filtration, washed with iso-propanol:water (1:1, 150 mL) and dried in vacuo at 40° C. for 21 hours to give the sub-title compound as a white crystalline solid (92.54 g, 87%). m.p. 73.5° C. ¹H-NMR (300 MHz, CDCl₃) δ1.42 (9H, s), 2.31 (3H, s), 2.62 (6H, s) 3.40 (2H, q), 4.01 (2H, t), 4.83 (1H, bs), 6.98 (2H, s)

(c) [2-(7-Benzyl-9-oxa-3,7-diazabicyclo[3.3.1]non-3-yl)ethyl]carbamic acid tert-butyl ester, 2,4,6-trimethylbenzenesulfonic acid salt

A warm (28° C.) solution of 2-(tert-butyloxycarbonylamino)ethyl 2,4,6-trimethylbenzenesulfonate (70.93 g, 206.03 mmole, 1.0 eq, see step (b) above) in toluene (240 mL, 4 vols) was added to a solution of 3-benzyl-9-oxa-3,7-diazabicyclo[3.3.1]nonane (44.98 g, 206.03 nunole, 1.0 eq.) in toluene (240 mL, 4 vols) (see step (a) above). The resultant solution was stirred rapidly under nitrogen, with heating at 68° C. for 8 hours. The reaction was left to stir at ambient temperature for 84 hours. A thick, white solid precipitate had formed in a pale yellow solution. The mixture was cooled to +9° C., and sub-title compound was collected by filtration. The reaction vessel was washed with toluene (100 mL) and added to the filter. The filter cake was washed with toluene (150 mL). The white solid product was suction dried for 15 minutes, then dried to constant weight in vacuo at 40° C. for 23 hours. The yield of sub-title compound obtained was 79.61 g, 141.7 mmole, 69%. The combined filtrate and washings (670 mL) were washed with aqueous sodium hydroxide solution (2M, 200 mL, 3.3 vols). The mixture was heated to 60° C., and held at this temperature for 20 minutes with rapid stirring. The two layers were then separated. The toluene solution was concentrated to 200 mL by vacuum distillation (bp 50-54° C. at 650-700 mbar; bp 46° C. at 120 mbar at the end). As the distillation progressed, the solution became cloudy due to the formation of sub-title compound. It was assumed that 20% of the original amount of 3-benzyl-9-oxa-3,7-diazabicyclo[3.3.1]nonane remained in the filtrate, and so extra 2-(tert-butyloxycarbonylamino)ethyl 2,4,6-trimethylbenzenesulfonate (14.20 g, 41.21 mmole, 0.2 eq) was added in one portion (charged as a solid rather than as a solution in toluene). The cloudy solution was heated at 67° C. for 8 hours with rapid stirring, and then left to stir at ambient temperature for 11 hours. The mixture was cooled to +8° C., and sub-title compound was collected by filtration. The reaction vessel was washed with more toluene (2×30 mL), and added to the filter. The white solid product was suction dried for 15 minutes, then dried to constant weight in vacuo at 40° C. for 7 hours. The yield of sub-title compound was 23.25 g, 41.39 mmole, 20%. The combined yield of sub-title compound (a white solid) was 102.86 g, 183.11 mmole, 89%. m.p. 190-190.5° C. ¹H-NMR (300 MHz, CDCl₃) δ1.43 (9H, s), 2.17 (3H, s), 2.51 (6H, s), 2.73- 2.80 (2H, m), 2.90-2.94 (4H, m), 3.14-3.22 (4H, m), 3.37 (2H, bm), 3.89 (2H, bs), 4.13 (2H, bs), 6.74 (2H, s), 7.12 (1H, bt), 7.42-7.46 (5H, m)

(ii) [2-(9-Oxa-3,7-diazabicyclo[3.3.1]non-3-yl)ethyl]carbamic acid tert-butyl ester

Method 1: Sodium bicarbonate (0.058 g, 0.069 mmol) and 5% Pd/C (0.250 g, Johnson Matthey Type 440 paste) were added to a solution of [2-(7-benzyl-9-oxa-3,7-diazabicyclo[3.3.1]non-3-yl)ethyl]carbamic acid tert-butyl ester (see step (i), Alternative 1 above; 1 g, 2.77 mmol) in ethanol (10 mL). The mixture was then hydrogenated at 500 kPa (5 bar) for 18 hours. The reaction mixture was filtered through Celite® and then washed with ethanol (20 mL). The solution was concentrated under reduced pressure to give an oil. This was dissolved in dichloromethane (20 mL) and washed with sodium hydroxide (1 M, 10 mL). The organic phase was separated, dried over magnesium sulfate and then filtered. The filtrate was concentrated under reduced pressure to give the sub-title compound as a yellow solid (0.67 g, 87%). m.p. 91-93° C. ¹H-NMR (300 MHz, CDCl₃) δ1.46 (9H, s), 2.25 (2H, t), 2.58-2.65 (2H, m) 2.95-3.06 (4H, m), 3.2-3.38 (4H, m), 3.64 (2H, bs), 4.65 (1H, bs). MS:m/z=272 (MH⁺).

Method 2: [2-(7-Benzyl-9-oxa-3,7-diazabicyclo[3.3.1]non-3-yl)ethyl]-carbamic acid tert-butyl ester 2,4,6-trimethylbenzenesulfonic acid salt (320 g, 1.0 mol eq, 1.0 rel vol/wt, see step (i), Alternative 3 above), toluene (640 mL, 2.0 vol) and aqueous sodium hydroxide (1M, 1.6 L, 5.0 vol) were stirred together for 15 minutes and the layers were then separated. The organic layer, containing [2-(7-benzyl-9-oxa-3,7-diazabicyclo[3.3.1]non-3-yl)ethyl ]carbamic acid tert-butyl ester, was diluted with ethanol (690 mL, 2.16 vol) and water (130 mL, 0.4 vol). Citric acid (32.83g, 0.3 mol eq) and 5% Pd/C (20.8 g, 0.065 wt eq of 61% water wet catalyst, Johnson Matthey type 440L) were added. The combined mixture was then hydrogenated under 4 bar of hydrogen pressure for 24 hours. The reaction was monitored by TLC, using a silica plate with mobile phase X:DCM (1:1 v/v; X is chloroform:methanol:concentrated ammonia 80:18:2 v/v). Visualisatiot was by UV light (254 mn) and by staining with aqueous potassium permanganate. This showed the complete disappearance of starting material and the appearance of the sub-title compound. The reaction mixture was filtered through ideselguhr and was washed with ethanol (590 mL, 1.84 vol). The resulting solution of sub-title compound (assumed 154.85 g, 100%) was used directly in a subsequent reaction.

Method 3: [2-(7-Benzyl-9-oxa-3,7-diazabicyclo[3.3.1]non-3-yl)ethyl]-carbamic acid tert-butyl ester 2,4,6-trimethylbenzenesulfonic acid salt (50 g, 1.0 mol eq., 1.0 rel vol/wt, see step (i), Alternative 3 above), toluene (100 mL, 2.0 vol) and aqueous sodium hydroxide (1M, 100 L, 2.0 vol) were stirred together for 20 minutes, then at 30° C. for 10 minutes, and the layers were then separated. The organic layer, containing [2-(7-benzyl-9-oxa-3,7-diazabicyclo[3.3.1]non-3-yl)ethyl]carbamic acid tert-butyl ester, was diluted with ethanol (100 mL, 2.0 vol.). To this was added a solution of citric acid (5.14 g, 0.3 mol eq) in water (5 mL, 0.1 vol), followed by 5% Pd/C (1.50 g, 0.03 wt eq of 61% water wet catalyst, Johnson Matthey type 440L). The combined mixture was then hydrogenated under 4 bar of hydrogen pressure for 24 hours. The reaction was monitored by TLC, using a silica plate with mobile phase X:DCM 1:1 v/v, (X is chloroform:methanol:concentrated ammonia 80:18:2 v/v). Visualisation was by UV light (254 nm) and by staining with aqueous potassium permanganate. This showed the complete disappearance of starting material and the appearance of the sub-title compound. The reaction mixture was basified with aqueous sodium hydroxide (10M, 8 mL, 0.9 mol eq), then filtered through kieselguhr. The filter-cake was washed with ethanol (100 mL, 2.0 vol). The resulting solution of sub-title compound (assumed 24.15 g, 100%) was used directly in a subsequent reaction.

(iii) Compound B

Method I

3-(4-Cyanoanilino)propyl-4-methylbenzenesulfonate (see Preparation A(ii) above; 0.30 g, 0.92 mmol) and potassium carbonate (0.2 g, 1.38 mmol) were added to a solution of [2-(9-oxa-3,7-diazabicyclo[3.3.1]non-3-yl)ethyl ]carbamic acid tert-butyl ester (see step (ii), Method 1 above; 0.250 g, 0.92 mmol) in ethanol (5 mL). The reaction mixture was heated to 70° C. for 10 hours before concentrating the mixture under reduced pressure. The residue was partitioned between ethyl acetate (20 mL) and sodium hydroxide (1 M, 10 mL). The aqueous phase was re-extracted with ethyl acetate (20 mL). The combined organic phases were concentrated under reduced pressure to give a yellow solid (0.290 g). The solid was dissolved in ethyl acetate (10 mL) and this solution washed with a solution of citric acid (0.250 g) in water (10 mL). The aqueous phase was separated, basified with sodium hydroxide (1 M, 10 mL) and extracted with ethyl acetate (2×10 mL). All organic phases were combined, dried over magnesium sulfate and then filtered (washing filtered solids with ethyl acetate (10 mL)). The filtrate was concentrated under reduced pressure to give a yellow solid (0.160 g). This was slurried in ethyl acetate (0.2 mL) and then filtered to give title compound (0.050 g, 12%). m.p 113-115° C. ¹H-NMR (400 MHz, DMSO-D₆) δ1.32 (9H, s), 1.7 (2H, qt), 2.20 (2H, t), 2.22-2.3 (4H, m), 2.38-3.1 (2H, m) 2.8-2.85 (4H, 1H), 3.05 (2H, q), 3.19 (2H, q), 3.79 (2H, bs), 6.47 (1H, t), 6.66 (2H, d), 6.69 (1H, t), 7.41 (2H, d). MS:m/z=430 (MH⁺).

Method II

To the solution of [2-(9-oxa-3,7-diazabicyclo[3.3.1]non-3-yl)ethyl]carbamic acid tert-butyl ester generated in step (ii) (Method 3) above (assumed 24.15 g, 1.0 mol eq., 1.0 wt./vol.) in a mixture of toluene (approx. 100 mL), ethanol (approx. 200 mL) and water (approx. 14 mL), was added anhydrous potassium carbonate (18.58 g, 1.5 mol eq.). Solid 3-(4-cyanoanilino)propyl benzenesulfonate (28.17 g, 1.0 mol eq., see Preparation A(ix), Method 2, step (a) above) was added and the combined mixture was heated to 70° C. for six hours. The reaction was monitored by TLC using a silica plate with mobile phase X:DCM 1:1 v/v (in which X is chloroform:methanol:concentrated ammonia 80:18:2 v/v). Visualisation was by UV light (254 nm) and by staining with aqueous potassium permanganate. This showed the complete disappearance of starting material and the appearance of the title compound. The reaction mixture was cooled, and the solvent was concentrated in vacuo. The residue was partitioned between toluene (200 mL) and water (200 mL). The layers were separated, and the organic phase was concentrated in vacuo to afford a yellow solid (38.6 g). This crude material was dissolved in iso-propanol (190 mL, 5.0 rel. vol.) at 60° C., and the hot solution was filtered. The filtrate was stirred, and left to cool to room temperature. A white solid crystallised. The mixture was cooled from room temperature to approximately 8° C. The product was collected by filtration and was washed with iso-propanol (50 mL, 2.0 vol.). The damp product was dried in vacuo at 40° C. to constant weight to give the title compound as a white crystalline solid (30.96 g, 81%).. m.p. 113.5° C. ¹H-NMR (400 MHz, CD₃OD) δ1.40 (9H, s), 1.81-1.90 (2H, m), 2.35-2.54 (8H, m), 2.93 (4H, t) 3.18-3.27 (4H, m), 3.87 (2H, bs), 6.66 (2H, d), 7.39 (2H, d) MS:m/z=(MH⁺, 430)

Preparation C

Preparation of Compound C

(i) 4-(4-Cyanophenyl)but-3-yn-1-ol

Potassium carbonate (376.7 g, 2.5 mol eq.) was dissolved in a mixture of 1,2-dimethoxyethane (DME, 1.2 L, 6 vol) and water (1.2 L, 6 vol). Palladium on charcoal (20 g, 0.01 mol eq., 10% Johnson Matthey type 87L, 60% water), triphenylphosphine (11.5 g, 0.04 mol eq.) and copper(I) iodide (4.2 g, 0.02 mol eq.) were added. 4-Bromobenzonitrile (200 g, 1 mol eq.) was then added, washing in with a mixture of DME (200 mL, 1 vol) and water (200 mL, 1 vol). This mixture was stirred rapidly under nitrogen for a minimum of thirty minutes. A solution of but-3-yn-1-ol (92.1 mL, 1.1 mol eq) in DME (200 mL, 1 vol) and water (200 mL, 1 vol) was added dropwise over five minutes. The combined mixture was then heated to 80° C. for three hours. The reaction was monitored by HPLC for the disappearance of arylbromide and the formation of sub-title compound. Once all of the starting material had been consumed, the reaction was cooled to 25° C. and filtered through kieselguhr. The filter cake was washed separately with toluene (1.6 L, 8 vol). The DME:water mixture was partially concentrated in vacuo to remove the majority of the DME. This was then partitioned with the toluene wash. The toluene layer was concentrated in vacuo to give sub-title alkyne as a yellow solid, which was dried in a vacuum oven overnight at 40° C. Yield 182.88 g, 97%. ¹H NMR (300 MHz, CDCl₃) Λ7.599-7.575 (d, J=7.2 Hz, 2H, CH), 7.501-7.476 (d, J=7.5 Hz, 2H, CH), 3.880-3.813 (q, 2H, CH₂), 2.751-2.705 (t, 2H, CH₂), 1.791-1.746 (t, 1H, OH) mp 79.6-80.5° C.

(ii) 4-(4-Hydroxybutyl)benzonitrile

4-(4-Cyanophenyl)but-3-yn-1-ol (40 g, 1 wt eq, see step (i) above) in ethanol (200 mL, 5 vol) and palladium on charcoal (20 g, 0.5 wt eq, 10% Johnson Matthey type 487, 60% water) were stirred rapidly under five bar hydrogen pressure for five hours. The reaction was monitored by HPLC for the disappearance of the starting material, and the formation of sub-title compound. The reaction was filtered through kieselguhr and washed with ethanol (80 mL, 2 vol). The ethanol solution was concentrated in vacuo to give sub-title alcohol as a yellow-brown oil. Yield 36.2g, 88.5%. ¹H NMR (300 MHz, CDCl₃) Λ7.550-7.578 (d, J=8.4 Hz, 2H), 7.271-7.298 (d, J=8.1 Hz, 2H), 3.646-3.688 (t, 2H), 2.683-2.733 (t, 2H), 1.553-1.752 (m, 4H) ¹³C NMR (300 MHz, CDCl₃) Λ148.04 (C), 132.16 (C), 119.1 (C), 109.64 (C), 62.46 (C), 35.77 (C), 32.08 (C), 27.12 (C).

(iii) 4-(4-Cyanophenyl)butyl toluenesulphonate

The sub-title compound was prepared by addition of toluenesulphonyl chloride to 4-(4-hydroxybutyl)benzonitrile (see step (ii) above).

(iv) tert-Butyl 7-[4-(4-cyanophenyl)butyl]-9-oxa-3,7-diazabicyclo[3.3.1]-nonane-3-carboxylate

A 2L three-necked flask was equipped with a magnetic stirrer, a thermometer and a reflux condenser. The flask was charged with a solution of 4-(4-cyanophenyl)butyl toluenesulphonate (72 g, 0.218 mol, see step (iii) above) in dimethylformamide (0.55 L). tert-Butyl 9-oxa-3,7-diazabicyclo[3.3.1]nonane-3-carboxylate hydrochloride (48.2, 0.182 mol, see Preparation B(I)(iv) above) was added, followed by potassium carbonate (62.9 g, 0.455 mol). The heterogeneous mixture was stirred for 22 hours at 85° C. TLC analysis indicated complete consumption of starting material. The reaction mixture was cooled to room temperature and diluted with water (0.5 L). The mixture was extracted with ethyl acetate (3×0.4 L) and the organic fractions were combined. After washing with water (2×200 mL) and brine (200 mL), the organic layer was dried with magnesium sulfate, filtered and concentrated under vacuum. The crude brown oil was purified by chromatography on silica gel, eluting with 3:2 hexanes/ethyl acetate affording 34 g (48% yield) of sub-title compound as an off-white solid.

(v) 4-[4-(9-Oxa-3,7-diazabicyclo[3.3.1]non-3-yl)butyl]benzonitrile

A 2L three-necked flask was equipped with a magnetic stirrer, a thermometer and an addition funnel. The flask was charged with tert-butyl 7-[4-(4-cyanophenyl)butyl]-9-oxa-3,7-diazabicyclo[3.3.1]-nonane-3-carboxylate (34 g, 88 mmol, from step (iv) above) and dichloromethane (440 mL). Trifluoroacetic acid (132 mL) was added slowly at room temperature. The solution was stirred for three hours at which point TLC analysis showed complete consumption of starting material. The contents were transferred to a single-necked flask and concentrated under vacuum. The residue was dissolved in dichloromethane (500 mL) and washed with saturated sodium bicarbonate solution. The aqueous layer was separated and extracted with dichloromethane (2×200 mL). The combined organic layers were washed with brine (200 mL), dried over magnesium sulfate and concentrated under vacuum to afford 25.8 g (100% yield) of sub-title compound as an off-white solid. The crude material was used in the next step without further purification.

(vi) Compound C

A 3L three-necked flask was equipped with a magnetic stirrer, a thermometer and a reflux condenser. The flask was charged with unpurified 4-[4-(9-oxa-3,7-diazabicyclo[3.3.1]non-3-yl)butyl]benzonitrile (25.8 g, 88 mmol, from step (v) above), dichloromethane (0.88 L) and tert-butyl 2-bromoethylcarbamate (see Preparation B(l)(i) above, 27.7 g, 123 mmol). Triethylamine (0.0197 L, 0.141 mol) was then added. The clear solution was refluxed for 12 hours under a nitrogen atmosphere and then cooled to room temperature. The progress of the reaction was monitored by TLC analysis and it was found to be complete at this point. The reaction mixture was transferred to a separating funnel and washed sequentially with water (200 mL), 15% aqueous sodium hydroxide (200 mL), water (200 mL), and brine (200 mL). The organic layer was dried over magnesium sulfate and concentrated under vacuum. The resulting yellow viscous oil was chromatographed on silica gel, eluting first with 9:1 dichloromethane/methanol, then with 9:1:0.02 dichloromethane/methanol/28% aqueous ammonium hydroxide to afford the title compound (25.1 g, 66% yield) as an off-white solid. The earlier fractions (5.1 g) from chromatography were found to contain a small amount of a less polar impurity (by TLC analysis) eluting with 9:1:0.05 dichloromethane/methanol/28% aqueous ammonium hydroxide) while the later factions (20 g) were one spot by TLC analysis. The earlier fractions (5.1 g) were combined with another lot of title compound (7.1 g, containing a slight impurity) and chromatographed on silica gel, eluting first with 19:1 dichloromethane/methanol, and then with 9:1 dichloromethane/methanol to afford a pale yellow powder (5.5 g). The powder was dissolved in dichloromethane (200 mL). The resulting solution was washed sequentially with 25% aqueous sodium hydroxide (50 mL), water (50 mL), and brine (40 mL). The material was then dried over magnesium sulfate and concentrated under vacuum to afford title compound as an off-white powder (5 g). The 20 g fraction was dissolved in dichloromethane (500 mL). The organic layer was washed sequentially with 25% aqueous sodium hydroxide (100 mL), water (100 mL), and brine (100 mL). The material was then dried over magnesium sulfate and concentrated under vacuum to afford title compound as an off-white powder (19 g). The lots were blended together.

Preparation D

Preparation of Compound D

(i) 4-[(2S)-Oxiranylmethoxy]benzonitrile

Potassium carbonate (414 g) and (R)-(-)-epichlorohydrin (800 mL) were added to a stirred solution ofp-cyanophenol (238 g) in 2.0 L MeCN and the reaction mixture was refluxed under an inert atmosphere for 2 h. The hot solution was filtered and the filtrate concentrated, giving a clear oil which was crystallised from di-iso-propyl ether giving the product in 90% yield.

(ii) tert-Butyl 7-[(2S)-3-(4-cyanophenoxy)-2-hydroxypropyl]-9-oxa-3,7-diazabicyclo[3.3.1]nonane-3-carboxylate

A 3L, three-necked flask equipped with a magnetic stirrer and a thermometer was charged with tert-butyl 9-oxa-3,7-diazabicyclo[3.3.1]nonane-3-carboxylate as its free base (53.7 g, 0.235 mol, obtained from the hydrochloride salt, see Preparation B(I)(iv) above), 4-[(2S)-oxiranylmethoxy]benzonitrile (41.2 g, 0.235 mol, see step (i) above), and a 10:1 (v/v) solution of 2-propanol/water (0.94 L). The mixture was stirred at 60° C. for 20 hours, during which time the starting materials were gradually consumed (assay by TLC analysis). The mixture was cooled and concentrated under vacuum to afford 100 g (>100% yield) of sub-title compound as white solid. The unpurified material was used in the next step.

(iii) 4-{[(2S)-2-Hydroxy-3-(9-oxa-3,7-diazabicyclo[3.3.1 ]non-3-yl)propyl]-oxy}benzonitrile

A 3L, three-necked flask equipped with a magnetic stirrer, a thermometer and an addition funnel was charged with unpurified tert-butyl 7-[(2S)-3-(4-cyanophenoxy)-2-hydroxypropyl]-9-oxa-3,7-diazabicyclo[3.3.1 ]nonane-3-carboxylate (100 g, from step (ii) above) and dichloromethane (1.15 L).

Trifluoroacetic acid (0.352 L) was added slowly at room temperature and the resulting solution was stirred for three hours, at which point TLC analysis showed complete reaction. The contents were transferred to a single-necked flask and concentrated under vacuum. The residue was dissolved in dichloromethane (1.2 L) and washed with saturated sodium bicarbonate. The aqueous layer was separated and extracted with dichloromethane (2×0.2 L). The combined organic layers were washed with brine (0.25 L), dried over magnesium sulfate and concentrated under vacuum to afford 73 g (>100% yield) of sub-title compound as an off-white solid. The unpurified material was used in the next step.

(iv) Compound D

Method I A 2L, three-necked flask was equipped with a magnetic stirrer, a thermometer and a reflux condenser. The flask was charged with unpurified 4-{[(2S)-2-hydroxy-3-(9-oxa-3,7-diazabicyclo[3.3. l]non-3-yl)propyl]-oxy }benzonitrile (73 g, from step (iii) above), dichloromethane (0.7 L) and tert-butyl 2-bromoethylcarbamate (see Preparation B(I)(i) above, 74 g, 0.330 mol). Triethylamine (52 mL, 0.359 mol) was then added. The clear solution was refluxed for 16 hours and then cooled to room temperature. The reaction mixture was transferred to a separating fimnel and washed sequentially with water (100 mL) and brine (100 mL). The organic layer was dried over magnesium sulfate, filtered and concentrated under vacuum. The resulting yellow viscous oil was purified by chromatography on silica gel, eluting first with 9:1 dichloromethane/methanol, then with 9:1:0.02 dichloromethane/methanol/28% aqueous ammonium hydroxide to afford an off-white foamy solid (40 g). The solid was dissolved in dichloromethane (200 mL) and washed sequentially with 20% aqueous sodium hydroxide (100 mL) and water (100 mL). The organic layer was dried over magnesium sulfate and concentrated under vacuum to afford title compound as an off-white solid (35.4 g, 67% yield in three steps).

Method II iso-Propanol (5 mL) and water (0.5 mL) were added to [2-(9-oxa-3,7-diazabicyclo[3.3.1]non-3-yl)ethyl]carbamic acid tert-butyl ester (see Preparation B(II)(ii), Method I above; 0.43 g, 1.6 mmol) and 4-[(2S)-oxiranylmethoxy]benzonitrile (0.280 g, 1.6 mmol, see step (i) above) was added. The mixture was heated at 66° C. for 19 hours (reaction was complete in 2 hours). The solvent was evaporated to dryness under reduced pressure to give the title compound as an off-white solid (0.71 g, 100%). ¹H-NMR (300 MHz, CDCl₃) δ1.41 (9H, s), 2.3-2.75 (6H, m), 2.75-3.0 (5H, m), 3.1-3.38 (3H, m), 3.88 (2H, s), 3.95-4.19 (3H, m), 5.85 (1H, bs), 6.99 (2H, d), 7.6 (2H, d). ¹H-NMR (300 MHz, DMSO-D₆) δ1.35 (9H, s), 2.12-2.59 (7H, m), 2.63- 2.78 (1H, m), 2.78-2.9 (4H, m), 3.2 (2H, q), 3.78 (2H, m), 4-4.1 (2H, m), 4.12-4.19 (1H, m), 5.3 (1H, bs), 6.61 (1H, t), 7.15 (2H, d), 7.76 (2H, d). MS:m/z=447 (MH⁺).

Method III: The solution of [2-(9-oxa-3,7-diazabicyclo[3.3.1]non-3-yl)ethyl ]carbamic acid tert-butyl ester generated in Preparation B(II)(ii), Method 2 above (assumed 154.85 g, 1.0 mol eq, 1.0 wt/vol) in a mixture of toluene (approx 640 mL), ethanol (approx 1280 mL) and water (approx 130 mL), was basified with aqueous sodium hydroxide (10M, 51 mL, 0.9 mol eq.). Solid 4-[(2S)-oxiranylmethoxy]benzonitrile (99.80g, 1.0 mol eq.; see step (i) above) was added and the combined mixture was heated to 70° C. for four hours. The reaction was monitored by TLC using a silica plate with mobile phase X:DCM 1:1 v/v (in which X is chloroform:methanol:concentrated ammonia 80:18:2 v/v). Visualisation was by UV light (254 nm) and by staining with aqueous potassium permanganate. This showed the complete disappearance of starting material and the appearance of the title compound. The reaction mixture was cooled, filtered through kieselguhr and washed through with ethanol (620 mL, 4.0 vol). This gave a solution of title compound (assumed 254.38 g, 100% th, 2.4 L, 1.0 wt/vol for reaction work up). This solution was charged into a flask that was set up for reduced pressure distillation. A graduation line was marked onto the side of this flask. Solvent (1250 mL) was removed at between 50° C. and 35° C., 320 mbar and 100 mbar. Then 4-methylpentan-2-ol (1500 mL) was added in order to reach the graduated line. Solvent (1250 mL) was removed at between 35° C. and 80° C., 220 mbar and 40 mbar. More 4-methylpentan-2-ol (1500 mL) was added in order to reach the graduated line. Solvent (1250 mL) was removed at between 62° C. and 76° C., 100 mbar and 90 mbar. The combined mixture was cooled to less than 25° C. and aqueous sodium hydroxide (2M, 1.27 L, 5.0 vol) was added. The layers were separated and the organic layer was filtered through kieselguhr to give a clear solution (1.2 L). This solution was charged into a clean flask, which was set up for reduced pressure distillation. Solvent (450 mL) was removed at between 52° C. and 55° C., 90 mbar and 35 mbar. Theoretically, the product was now left in 2 volumes of 4-methylpentan-2-ol. Di-n-butyl ether (1.27 L, 5 vol) was added and the solution was allowed to cool slowly to room temperature, which caused a precipitate to form. The mixture was cooled from room temperature to approximately 10° C. The product was collected by filtration and was washed with a pre-mixed solution of di-n-butyl ether (320 mL, 1.25 vol) and 4-methylpentan-2-ol (130 mL, 0.50 vol). 2s The danp product was dried in vacuo at 55° C. to constant weight to give the title compound as a white solid (193.6 g, 76%). m.p. 99-101° C. ¹H-NMR (300 MHz, CDCl₃) δ1.41 (9H, s), 2.3-2.75 (6H, m), 2.75-3.0 (5H, m), 3.1-3.38(3H, m), 3.88 (2H, s), 3.95-4.19 (3H, m), 5.85 (1H, bs), 6.99 (2H, d), 7.6 (2H, d).

Crystallisation of Compound D

A mixture of Compound D (prepared analogously to the procedures described hereinbefore (see especially Preparation D(iv), Method III above); 14.29 g), iso-propanol (28 mL) and di-iso-propyl ether (140 mL) was heated to 80° C. The solution was filtered hot to clarify it and then reheated to 80° C. The solution was then allowed to cool to room temperature whereupon a precipitate started to form. After stirring for two hours the precipitate was collected by filtration, washed with iso-propanol:iso-propyl ether (1:6, 70 mL) and then sucked dry on the filter. The damp product was dried in vacuo at 70° C. overnight to give crystalline Compound D as a white solid (10.1 g, 70%/). ¹H-NMR (300 MHz, CDCl₃) δ1.41 (9H, s), 2.3-2.75 (6H, m), 2.75-3.0 (SH, m), 3.1-3.38(3H, m), 3.88 (2H, s), 3.95-4.19 (3H, m), 5.85 (1H, bs), 6.99 (2H,.d), 7.6 (2H, d)

Preparation of Other Salts of Compound A

para-Toluenesulphonic acid, 1-hydroxy-2-naphthoic acid, 1,5-naphthalene-sulphonic acid and 2-mesitylenesulphonic acid salts of Compound A were prepared by dissolving Compound A (prepared using analogous techniques to those described in Preparation A described above) in ethyl acetate and adding a solution of the appropriate acid in methanol, followed by standard work up and isolation. Benzoic acid, para-hydroxybenzenesulphonic acid and 1,5-naphthalenedisulphonic acid salts were prepared in a similar fashion.

Preparation of Salts of Compound C

Methanesulphonic acid and para-toluenesulphonic acid salts of Compound C were prepared by dissolving Compound C (prepared using analogous techniques to those described above) in methanol and adding, directly, the appropriate acid, followed by standard work up and isolation.

Preparation of Salts of Compound D

Methanesulphonic acid and hippuric acid salts of Compound D were prepared by dissolving Compound D (prepared using analogous techniques to those described above) in methanol. and adding the appropriate acid (directly in the case of methanesulphonic acid and as a solution in methanol in the case of hippuric acid), followed by standard work up and isolation. The methanesulphonic acid salt was also prepared by dissolving Compound D in ethyl acetate and adding methanesulphonic acid as a solution in ethyl acetate, followed by seeding, standard work up and isolation. 1,5-Napthalenedisulphonic acid, terephthalic acid, succinic acid, O,O′-di-para-toluoyl-D-tartaric acid and pamoic acid salts were prepared in a similar fashion. A hemisuccinic acid salt of Compound D was prepared by dissolving Compound D and succinic acid in isopropanol, followed by seeding, standard work up and isolation. O,O′-dibenzoyl-D-tartaric acid, 2,2,3,3-tetramethyl-1,4-dibutanoic acid and 1,2-cyclopentanedi-carboxylic acid salts were prepared by dissolving Compound D in ethyl acetate and adding the appropriate acid as a solution in methanol, co-evaporation of solvents, addition of further ethyl acetate, crystallisation, standard work up and isolation.

Compound D, [(biphenyl-4-carbonyl)amino]acetic acid salt was prepared as follows:

(a) [(Biphenyl-4-carbonyl)amino]acetic acid methyl ester

Dichloromethane (50 mL) and then triethylamine (11.2 mL, 79.6 mmol, 2.0 eq) were added to glycine methyl ester hydrochloride (5.0 g, 39.8 mmol, 1.0 eq). The mixture was stirred and cooled to −5° C. using an ice/methanol bath. A suspension of biphenyl-4-carbonyl chloride (8.26 g, 39.8 mmol, 1.0 eq) in dichloromethane (25 mL) was added over 22 minutes. The mixture was stirred for 3 hours at −5° C., and then left to stir at room temperature overnight (16 hours). Water (75 mL) was added and the mixture was stirred rapidly for 30 minutes at room temperature. The layers were separated. The organic layer was washed with water (75 mL), then evaporated to dryness using a rotary evaporator to give an off-white solid (6.58 g, 62%). ¹H-NMR (300 MHz, CDCl₃) δ3.82 (s, 3H), 4.29 (d, J =5.1 Hz, 2H), 6.68 (s, 1H), 7.3-7.5 (m, 3H), 7.62 (d, J=4.8 Hz, 2H), 7.68 (d, J=8.1 Hz, 2H), 7.90 (d, J=8.4Hz, 2H) m.p.127-128° C.

(b) [(Biphenyl-4-carbonyl)amino]acetic acid [(Biphenyl-4-carbonyl)amino]acetic acid methyl ester (6.58 g, 25 mmol, 1.0 eq., from step (a) above) was added to the flask followed by aqueous sodium hydroxide (1M, 84 mL, 50 mmol, 2.0 eq). The mixture was heated to 50° C. using an oil bath for 5 hours. The solution was then stirred overnight (16 hours) at room temperature. On cooling, a white precipitate formed. The mixture was cooled further to 5° C. using an ice/water bath. Concentrated hydrochloric acid (8 mL) was added very slowly to the cooled solution, ensuring that the temperature did not rise above 10° C. The mixture was stirred for 15 minutes and was then filtered. The white solid was air dried for 30 minutes and then dried in vacuo at 40° C. for 16 hours to give an off-white solid (5.75 g, 93%). ¹H-NMR (300 MHz, DMSO-d₆) δ3.95 (d, J=5.7 Hz, 2H), 7.35-7.5 (m, 3H), 7.7-7.8 (m, 4H), 7.97 (d, J=6.9 Hz, 2H), 8.89 (t, J =6.0 Hz, 1H), 12.58 (s, 1H) mp 217-217.5° C.

(c) Recrystallisation of [(biphenyl-4-carbonyl)amino]acetic acid

Methanol (100 mL, 20 vols) was added to [(biphenyl-4-carbonyl)amino]-acetic acid (5.0 g, from step (b) above). The mixture was heated to 62° C. using an oil bath whilst being stirred. The resulting pale orange solution was held at this temperature for 10 minutes. The solution was allowed to cool to room temperature, and then was cooled further to 5° C. using an ice/water bath. Crystallisation began at approximately 30° C. The precipitate was collected by filtration, air dried for 15 minutes, then dried in vacuo at 40° C. for 26 hours to give colourless crystals (2.9 g, 58 %). ¹H-NMR (300 MHz, DMSO-d₆) δ3.95 (d, J=5.7 Hz, 2H), 7.35-7.5 (m, 3H), 7.7-7.8 (m, 4H), 7.97 (d, J=6.9 Hz, 2H), 8.89 (t, J=6.0 Hz, 2H), 12.58 (s, 1H)

(d) Compound D, [(biphenyl-4-carbonyl)amino]acetic acid salt

[(Biphenyl-4-carbonyl)amino]acetic acid (1.14 g, see steps (b) or (c) above) and Compound D (2 g, prepared analogously to methods described hereinbefore) were dissolved in hot iso-propanol (40 mL). On cooling to room temperature a crystalline precipitate formed which was filtered, washed with iso-propanol (2×20 mL) and sucked dry on the filter. Drying for 6 hours in vacuo at 40° C. gave the salt as a colourless, crystalline solid (2.50 g, 80%). ¹H-NMR (300 MHz, DMSO-d₆) δ1.34 (9H, s), 2.25 (2H, t), 2.3-2.5 (4H, m), 2.6-2.7 (1H, m), 2.7-2.8 (1H, m), 2.85-3.0 (4H, m), 3.0-3.1 (2H, m), 3.82 (2H, s), 3.88 (2H, d), 3.95-4.05 (2H, m), 4.1-4.2 (1H, m), 6.65 (1H, t), 7.14 (2H, d), 7.35-7.55 (3H, m), 7.7-7.85 (6H, m), 7.96 (2H, d), 8.75 (1H, t) mp 143-143.5° C.

Compound D, (3,4-dichlorobenzoylamino)acetic acid salt was prepared as follows:

(a) (3,4-Dichlorobenzoylamino)acetic acid methyl ester

Dichloromethane (150 mL) and then triethylamine (33.0 mL, 234 mmol, 2.0 eq.) were added to glycine methyl ester hydrochloride (14.7 g, 117 mmol, 1.0 eq.). The mixture was stirred and cooled to 2° C. using an ice/water bath. A solution of of 3,4-dichlorobenzoyl chloride (24.55 g, 117 mmol, 1.0 eq.) in dichloromethane (75 mL) was added over 7 minutes. The mixture was stirred for 1 hour at 2° C., then left to stir at room temperature overnight (16 hours). Water (225 mL) was added and the mixture was stirred rapidly for 30 minutes at room temperature. The layers were separated. The organic layer was washed with water (225 mL), then evaporated to dryness using a rotary evaporator to give an off-white solid. The isolated solid (26.18 g, 85%) was added to dichloromethane (300 mL, 10 vols.) with 1M sodium hydroxide solution (300 mL, 10 vols). The lower organic layer was concentrated to dryness in vacuo (25.91 g, 84%). m.p. 133.2-134.3° C. δ_{H} (300 MHz, CDCl₃) 3.66 (1H, s, CH₃), 4.03 (2H, d, J=6, CH₂), 7.78-7.87 (2H, m, CH), 8.100 (1H, s, CH), 9.18 (1H, t, J=5.7, NH).

(b) (3,4-Dichlorobenzoylamino)acetic acid

(3,3-Dichlorobenzoylamino)acetic acid methyl ester (25.91 g, 100 mmol, 1.0 eq., see step (a) above) was added to the flask followed by aqueous sodium hydroxide (1M, 198 mL, 200 mmol, 2.0 eq.). The mixture was heated to 50° C. using an oil bath for 2 hours. On cooling, a white precipitate formed. The mixture was cooled further to 5° C. using an ice/water bath. Concentrated hydrochloric acid (60 mL) was added very slowly to the cooled solution, ensuring that the temperature did not rise above 10° C. The mixture was stirred for 10 minutes and was then filtered. The white solid was air dried for 15 minutes and then dried in vacuo at 40° C. for 16 hours to give an off-white solid (19.15 g, 78%). m.p. 140.0 - 140.3° C. δ_{H} (300 MHz, DMSO-D₆) 3.94 (2H, d, J=6, CH₂), 7.77- 7.87 (2H, m, CH), 8.10 (1H, s, CH), 9.06 (1H, t, J=6), 12.66 (1H, bs, OH)

(c) Compound D, (3,4-dichlorobenzoylamino)acetic acid salt

(3,4-Dichlorobenzoylamino)acetic acid (0.56 g, see step (b) above) and Compound D (1.02 g; prepared analogously to procedures described hereinbefore) were dissolved in hot ethyl acetate (4 mL). On cooling to room temperature, a crystalline precipitate formed which was filtered, washed with ethyl acetate (15 mL) and sucked dry on the filter. Drying overnight in vacuo at 40° C. gave the title salt as a colourless, crystalline solid (0.92 g, 58%). m.p. 128.5-130.5° C. ¹H-NMR (400 MHz, DMSO-D₆) δ1.34 (9H, s), 2.26 (2H, t), 2.3-2.5 (3H, m), 2.5-2.6 (1H, m), 2.6-2.7 (1H, m), 2.7-2.8 (1H, m), 2.85-3.0 (4H, m), 3.0-3.1 (2H, m), 3.8-3.9 (4H, m), 4.01 (2H, d), 4.1-4.2 (1H, m), 6.69 (1H, t), 7.12 (2H, d), 7.7-7.8 (3H, m), 7.84 (1H, dd), 8.09 (1H, dd) 8.92 (1H, t)

Compound D, [(naphthalene-2-carbonyl)amino]acetic acid salt was prepared as follows:

(a) [(Naphthalene-2-carbonyl)amino]acetic acid methyl ester

Dichloromethane (66 mL) and then triethylamine (14.6 mL, 105 mmol, 2.0 eq.) were added to glycine methyl ester hydrochloride (6.61 g, 52.5 mmol, 1.0 eq.). A white precipitate appeared on the addition of the triethylamine, and the solution became a lot thicker. The mixture was stirred and cooled to 2° C. using an ice/water bath. A solution of 2-naphthoyl chloride (10.07 g, 52.5 mmol, 1.0 eq.) in dichloromethane (33 mL) was added over 15 minutes. The pale brown mixture was stirred for 25 hours at 5° C. Water (100 mL) was added and the mixture was stirred rapidly for 30 minutes at room temperature. The layers were separated. The organic layer was washed with sodium hydroxide (1M, 100 mL) and then evaporated to dryness using a rotary evaporator to give an off-white solid (12.21 g, 96%). m.p. 117.7- 118.1° C. δ_{H} (400 MHz, DMSO-D₆) 3.68 (3H, s, CH₃), 4.08 (2H, d, J=4.5, CH₂), 7.59-7.66 (2H, m, CH), 7.935- 8.015 (4H, m, CH), 8.491 (1H, s, CH), 9.124 (1H, t, J=45.6, NH)

(b) [(Naphthalene-2-carbonyl)amino]acetic acid

[(Naphthalene-2-carbonyl)amino]acetic acid methyl ester (10.03 g, 41 mmol, 1.0 eq., see step (a) above) was added to the flask followed by aqueous sodium hydroxide (1M, 120 mL, 123 mmol, 3.0 eq.). The mixture was heated to 55° C. using an oil bath for 2 hours. The mixture was cooled to 5° C. using an ice/water bath. Concentrated hydrochloric acid (50 mL) was added very slowly to the cooled solution, ensuring that the temperature did not rise above 10° C. A dense yellow precipitate was formed. The mixture was stirred for 10 minutes and was then filtered. The yellow solid was air dried for 15 minutes and then dried in vacuo at 40° C. for 16 hours (8.73 g, 93%). Methanol (50 mL, 10 vols) and water (100 ml, 20 vols) were added to a portion of the sub-title compound (5.0 g, 22 mmol). The mixture was heated to 70° C. using an oil bath whilst being stirred. The solution was held at this temperature for 10 minutes, and then was allowed to cool further to 5° C. using an ice/water bath. Crystallisation began at approximately 30° C. The precipitate was collected by filtration, air dried for 15 minutes, then dried in vacuo at 40° C. for 2 hours (3.2. g, 64%). The isolated sub-title compound (3.2 g, 0.014 mol, 64%) was added to water (100 mL, 20 vols) and methanol (50 mL, 10 vols). The mixture was heated to 70° C. to dissolve the solid. The solution was allowed to cool to room temperature, crystallisation occurred on cooling. The mixture was cooled further to 2° C., and then was filtered using a sinter finnel. The solid was air dried for 10 minutes, then dried in vacuo at 40° C. for 16 hours (2.21 g, 44 %). m.p.167.1-167.4° C. δ_{H} (400 MHz, DMSO-D₆) 3.98 (2H, d, J=5.6, CH₂), 7.58-7.65 (2H, m, CH), 7.95- 8.05 (4H, m, CH), 8.49 (1H, s, CH), 8.99 (1H, t, J=5.6, NH), 12.63 (1H, bs, OH)

(c) Compound D, [(naphthalene-2-carbonyl)amino]acetic acid salt

[(Naphthalene-2-carbonyl)amino]acetic acid (0.51 g, see step (b) above) and Compound D -(1.01 g; prepared analogously to procedures described hereinbefore) were dissolved in methyl iso-butyl ketone (30 mL) at 100° C. On cooling to room temperature a crystalline precipitate formed which was filtered, washed with acetone (25 mL) and sucked dry on the filter. Drying over a weekend in vacuo at 40° C. gave the title salt as a colourless, crystalline solid (1.17 g, 77%). m.p. 138.5-140° C. ¹H-NMR (300 MHz, DMSO-D₆) δ1.34 (9H, s), 2.25 (2H, t), 2.3-2.5 (4H, m), 2.6-2.7 (1H, m), 2.7-2.8 (1H, m), 2.85-3.0 (4H, m), 3.0-3.1 (2H, m), 3.81 (2H, s), 3.92 (2H, d), 3.95-4.05 (2H, m), 4.1-4.2 (1H, m), 6.68 (1H, t), 7.11 (2H, d), 7.5-7.7 (2H, m), 7.7-7.8 (2H, m), 7.9-8.1 (4H, m), 8.47 (1H, d), 8.85 (1H, t).

Tablet Manufacture

Tablets were manufactured using a standard tabletting machine (Kilian SP300) in accordance with standard procedures.

Where appropriate, mixtures of polymer, drug and, if present, other excipients, were dry mixed (for example in a mortar) or wet or dry granulated using standard techniques. In relation to ethanol and water granulation on a small scale, active ingredient, polymer and, if appropriate, further excipient were dry mixed together in a mortar. An appropriate quantity of solvent was added with mixing. The granulate was dried at 50° C. for 16 hours.

Test Method

Drug/time release profiles for the tablets were determined using a United States Pharmacopoeia Method II (European Pharmacopoeia Paddle Method) apparatus with a UV detector and a paddle speed of 50 rpm (unless otherwise specified). A basket (see Int. J. Pharm., 60 (1990) 151) containing the tablet was placed 1 cm above the paddle. The release medium was phosphate buffer (pH=6.8) or HCl (pH=1.0). The temperature in the release bath was 37° C. The volume of the release medium was 1000 mL, unless otherwise specified.

Materials

Unless otherwise specified, HPMC polymers were obtained from Shin-Etsu (trademark METOLOSE™). Specific grades and their USP equivalents are indicated below (once only, on the first occasion that they are disclosed).

### EXAMPLE 1

HPMC (65SH1500; eq. to USP HPMC 2906, 1500 cps) was dry mixed together with Compound A (free base and benzenesulphonate salt thereof) in a weight ratio of 1:1. Tablets (diameter 10 mm) were made by direct compression using the Kilian SP300. The final tablet weight was about 250 mg. Drug release profiles were determined (pH 1.0 and 6.8) and are shown in FIGS. 1(a) and 1(b).

### EXAMPLE 2

Polymers (HEC (NATRASOL®250M Pharm; Aqualon) and PEO (MW 4 ×10⁶ g/mol; POLYOX® Union Carbide) were individually dry mixed together with Compound A (free base and benzenesulphonate salt thereof) in a weight ratio of 1:1. Tablets (diameter 10 mm) were made using the Kilian SP300. The final tablet weight was about 250 mg. The HEC tablets were coated with HPMC (viscosity 6 cps) by placing them in a 10% HPMC (eq. to USP HPMC 2910, 6 cps) solution in water and drying in air at room temperature. Drug release profiles were determined (pH 1.0 and 6.8) and are shown in FIGS. 2(a) to 2(d).

### EXAMPLE 3

Separate batches of the benzenesulphonate salt of Compound A (45 mg/tablet), HPMC (65SH400; eq. to USP HPMC 2906, 400 cps; 35 mg/tablet), calcium phosphate (10 mg/tablet), polyvinylpyrrolidone (PVPK90 (BASF); 8 mg/tablet) and PRUV® (sodium stearyl fumarate; Penwest Pharmaceuticals; 2 mg/tablet) were dry mixed together. For the first batch, tablets were made via direct compression using the Kilian SP300 of the dry mixed material. For the second batch, the dry mixture was ethanol granulated and dried. For the third batch, the dry mixture was water granulated and dried. Granules were then compressed using Kilian SP300. The tablet weight was about 100 mg in each case. Drug release profiles were determined for the three batches (pH 6.8) and are shown in FIG. 3.

### EXAMPLE 4

HPMC with different molecular weights (65SH50 (eq. to USP HPMC 2906, 50 cps), 65SH400 and 65SH1500), and/or different degrees of substitution (60SH50 (eq. to USP HPMC 2910, 50 cps), 65SH50 and 90SH1OO (eq. to USP HPMC 2208, 100 cps), were dry mixed together with the benzenesulphonate salt of Compound A in a weight ratio of 1:1. Tablets (with a diameter of 10 mm) were made using the Kilian SP300. The tablet weight was about 250 mg. Drug release profiles were determined for formulations with different degrees of substitution (pH 1.0 (see FIG. 4(a) and pH 6.8 (see FIG. 4(b))) and for formulations with different molecular weights (pH 6.8; see FIG. 4(c)).

### EXAMPLE 5

HPMC (60SH10000; eq. to USP HPMC 2910, 10,000 cps) was dry mixed together with the benzenesulphonate salt of Compound A in different weight ratios (25% salt, 60% salt and 75% salt). Tablets were direct compressed using the Kilian SP300. The final tablet weights were about 90 mg in each case. Drug release profiles were determined (paddle speed of 25 rpm; pH 6.8) and are shown in FIG. 5.

### EXAMPLE 6

HPMCs with different molecular weights (60SH50 and 60SH10000) were dry mixed together in weight ratios of 1:0, 1:2, 2:1 and 0:1. These combinations were dry mixed together with the benzenesulphonate salt of Compound A. The mixture was granulated using water (about 40% water to the dry total weight) and dried. Tablets (diameter 8.5 mm) were made using the Kilian SP300. The final tablet weight was about 175 mg. Thus, the dose of drug in the form of salt was 70 mg. Drug release profiles were determined (pH 6.8) and are shown in FIG. 6. In this case, the volume of the release medium was 500 mL.

### EXAMPLE 7

HPMC (65SH1500) was dry mixed together with Compound A (free base and benzenesulphonate salt thereof) in a weight ratio of 1:1. Tablets (diameter 20 mm) were made using the Kilian SP300. The final tablet weight was about 1000 mg. The dose of drug (free base or salt) was 560 mg. Drug release profiles were determined (pH 6.8) and are shown in FIG. 7.

### EXAMPLE 8

52.5 g of one grade of HPMC (METHOCEL™ K100LV CR grade, eq. to USP HPMC 2208, 100 cps, Dow), 78.7 g of another grade of HPMC (METHOCEL™ K4M grade, eq. to USP HPMC 2208, 4000 cps, Dow) and 87.5 g of the benzenesulphonate salt of Compound A were dry mixed together in a mixer (Braun CombiMax 750) with four blades on the impellers. 108.0 g of water was sprayed through a nozzle into the mixer (25 mL/minutes). The granulate was dried using a fluid bed (Glatt GPCG 1) using a bed speed of 50 m3/h and a insert temperature of 60° C. The fluid bed was turned off after about 14 minutes. At this point, the temperature in the bed was 47° C. The dry granulate was passed through a sieve (1 mm) and mixed with 1.93 g sodium stearyl fumarate in a food processor (the sodium stearyl fumarate was pre-sieved using a 1 mm sieve). Tablets were made from the lubricated granulate using a tabletting machine with 6 stations (Korsch PH 106-3). The tablet shape was concaved, and the size was 8 mm in diameter and about 4 mm in height. The weight was 184 mg. Drug release profiles were determined (pH 6.8) and are shown in FIG. 8.

### EXAMPLE 9

HPMC (65SH50) was dry mixed together with Compound D (free base) in a weight ratio of 1:1. Tablets (diameter 10 mm) were made by direct compression using the Kilian SP300. The final tablet weight was about 250 mg. Drug release profiles were determined (pH 1.0 and 6.8) and are shown in FIG. 9.

### EXAMPLE 10

120 mg of HPMC (60SH50), and 120 mg of HPMC (60SH10000) were dry mixed together with 10 mg of Compound D (free base). Tablets (diameter 10 mm) were made by direct compression using the Kilian SP300. The final tablet weight was about 250 mg. Drug release profiles were determined (pH 1.0 and 6.8) and are shown in FIG. 10.

### EXAMPLE 11

HPMC polymers with different molecular weights (60SH50 and 60SH10000) were dry mixed together in a weight ratio of 3:1. This resultant polymer blend was dry mixed together with Compound D (free base), as well as with the following salts of Compound D: the hemisuccinate, the methanesulphonate, the (3,4-dichlorobenzoylamino)-acetate and the (+)-O,O′-di-para-toluoyl-D-tartrate (prepared as described hereinbefore). Tablets (diameter 8 mm) for each individual combination were made by direct compression using the Kilian SP300. The final tablet weight was about 125 mg. The dose of the drug was 10 mg (with respect to the free base). Drug release profiles were determined (pH 6.8) and are shown in FIG. 11.

### EXAMPLE 12

HPMC (60SH10000) was dry mixed with Compound D, in the form of its free base as well as the following salts of Compound D: the hemisuccinate, the methanesulphonate and the (+)-O,O′-di-para-toluoyl-D-tartrate, in a weight ratio of 60:40 (polymer:drug). Tablets (diameter 8 mm) for each individual combination were made by direct compression using the Kilian SP300. The tablet weights varied between 125 mg and 178.8 mg depending on the different molecular weight of the base and the salts. The dose of drug was 50 mg (with respect to the free base). Drug release profiles were determined (pH 6.8) and are shown in FIG. 12.

### EXAMPLE 13

HPMC (60SH1000) was dry mixed with Compound D (free base) in the following weight ratios: 90:10, 80:20, 70:30, 60:40, 50:50, 40:60 and 30:70. Tablets (diameter 8 mm) were made by direct compression using the Kilian SP300. The final tablet weight was about 125 mg. The dose of drug varied between 12.5 mg and 87.5 mg. Drug release profiles were determined (pH 6.8) and are shown in FIG. 13.

### EXAMPLE 14

HPMC (60SH10000) was dry mixed with Compound D (free base) in weight ratios of 96:4, 70:30, 60:40 and 50:50. Tablets (diameter 12 mm) were made by direct compression using the Kilian SP300. The final tablet weights were about 625 mg. The dose of drug varied between 25 mg and 187.5 mg. Drug release profiles were determined (pH 6.8) and are shown in FIG. 14.

### EXAMPLE 15

HPMC (60SH10000) was dry mixed with Compound D (free base) in weight ratios of 37.5:62.5, 53.3:46.7, 60:40, 61.8:38.2, 66.7:33.3, 69.7:30.3, 78.3:21.7, 80:20 and 83.3:16.7. Tablets (diameter 8 mm) were made by direct compression using the Kilian SP300. The final tablet weights varied between 80 mg and 300 mg. Drug release profiles were determined (pH 6.8) and are shown in FIG. 15.

### EXAMPLE 16

Xanthan gum (XANTURAL® 180; CPKelco) was dry mixed with Compound D (free base) in weight ratios of 90:10, 80:20, 70:30 and 60:40. Tablets (diameter 8 mm) were made by direct compression using the Kilian SP300. The final tablet weight was about 125 mg. The dose of Compound D (free base) varied between 12.5 mg and 50 mg. Drug release profiles were determined (pH 6.8) and are shown in FIG. 16.

### EXAMPLE 17

375 mg of xanthan gum (KELTROL® RD; CPKelco) was dry mixed with 250 mg of Compound D (free base). Tablets (diameter 12 mm) were made by direct compression using the Kilian SP300. The final tablet weight was 625 mg. Drug release profiles were determined (pH 6.8) and are shown in FIG. 17.

### EXAMPLE 18

Xanthan gum (XANTURAL® 180; CPKelco) was dry mixed with Compound D (free base) in ratios of 40:60, 33.3:66.7, 25:75 and 20:80. Tablets (diameter 8 mm) were made by direct compression using the Kilian SP300. The final tablet weight varied between 125 mg and 150 mg. Drug release profiles were determined (pH 6.8) and are shown in FIG. 18.

### EXAMPLE 19

HPMC (60SH10000) was dry mixed with the methanesulphonic acid salt of Compound D in weight ratios of 30.4:121.6, 45.6:106.4 and 60.8:91.2. Tablets (8 mm) were made by direct compression using the Kilian SP300. The final tablet weight was 152 mg. Drug release profiles were determined (pH 1.0 and pH 6.8) and are shown in FIG. 19.

### EXAMPLE 20

HPMC (60SH10000) was dry mixed with the methanesulphonic acid salt of Compound D in weight ratios of 228:532, 304:456 and 380:380. Tablets (12 mm) were made by direct compression using the Kilian SP300. The final tablet weight was 760 mg. Drug release profiles were determined (pH 1.0 and pH 6.8) and are shown in FIG. 20.

Abbreviations

API=atmospheric pressure ionisation (in relation to MS)

br=broad (in relation to NMR)

d=doublet (in relation to NMR)

DCM=dichloromethane

DMF=N,N-dimethylformamide

DMSO=dimethylsulfoxide

dd=doublet of doublets (in relation to NMR)

Et=ethyl

eq.=equivalents

GC=gas chromatography

h=hour(s)

HCl=hydrochloric acid

HPLC=high performance liquid chromatography

IMS=industrial methylated spirit

IPA=iso-propyl alcohol

KF=Karl-Fischer

m=multiplet (in relation to NMR)

Me=methyl

MeCN=acetonitrile

min.=minute(s)

m.p.=melting point

MS=mass spectroscopy

Pd/C=palladium on carbon

q=quartet (in relation to NMR)

rt=room temperature

s=singlet (in relation to NMR)

t=triplet (in relation to NMR)

TLC=thin layer chromatography

UV=ultraviolet

Prefixes n-, s-, i-, t- and tert- have their usual meanings: normal, secondary, iso, and tertiary.

1. A modified release pharmaceutical composition comprising, as active ingredient, 4-({3-[7-(3,3-dimethyl-2-oxobutyl)-9-oxa-3,7-diazabicyclo-[3.3.1]non-3-yl ]propyl}amino)benzonitrile, tert-butyl 2-{7-[3-(4-cyano-anilino)propyl ]-9-oxa-3,7-diazabicyclo[3 .3.1]non-3-yl}ethylcarbamate, tert-butyl 2-{7-[4-(4-cyanophenyl)butyl]-9-oxa-3,7-diazabicyclo[3.3.1]non-3-yl }ethylcarbamate, or tert-butyl 2-{7-[(2S)-3-(4-cyanophenoxy)-2-hydroxypropyl ]-9-oxa-3,7-diazabicyclo[3.3.1 ]non-3-yl}ethylcarbamate, or a pharmaceutically-acceptable salt of any of these compounds.

2. A modified release pharmaceutical composition comprising, as active ingredient, 4-({3-[7-(3,3-dimethyl-2-oxobutyl)-9-oxa-3,7-diazabicyclo-[3.3.1]non-3-yl ]propyl}amino)benzonitrile, tert-butyl 2-{7-[3-(4-cyano-anilino)propyl ]-9-oxa-3,7-diazabicyclo[3.3.1 ]non-3-yl}ethylcarbamate, tert-butyl 2-{7-[4-(4-cyanophenyl)butyl]-9-oxa-3,7-diazabicyclo[3.3.1]non-3-yl }ethylcarbamate, or tert-butyl 2-{7-[(2S)-3-(4-cyanophenoxy)-2-hydroxypropyl ]-9-oxa-3,7-diazabicyclo[3.3.1]non-3-yl}ethylcarbamate, or a pharmaceutically-acceptable salt of any of these compounds, and a pharmaceutically-acceptable carrier and/or other means, which carrier or means (as appropriate) gives rise to a modified release of active ingredient.

3. A composition as claimed in claim 1 or claim 2, wherein the active ingredient is provided together with a pharmaceutically-acceptable carrier.

4. A composition as claimed in any one of the preceding claims, wherein the composition is adapted to provide delayed and/or sustained release of active ingredient.

5. A composition as claimed in claim 4, wherein the release is sustained.

6. A composition as claimed in any one of the preceding claims, which is adapted for oral administration.

7. A composition as claimed in any one of the preceding claims, in which the active ingredient is embedded in a polymer matrix.

8. A composition as claimed in claim 7 (as dependent on claim 6), which is in the form of a gelling matrix modified-release system comprising a hydrophilic gelling component and active ingredient.

9. A composition as claimed in any one of the preceding claims, wherein the active ingredient is 4-({3-[7-(3,3-dimethyl-2-oxobutyl)-9-oxa-3,7-diazabicyclo-[3.3.1]non-3-yl ]propyl}amino)benzonitrile or a pharmaceutically-acceptable salt thereof.

10. A composition as claimed in claim 9, wherein the active ingredient is provided in the form of a benzenesulphonic acid salt or a toluenesulphonic acid salt.

11. A composition as claimed in claim 10, wherein the active ingredient is provided as 4-({3-[7-(3,3-dimethyl-2-oxobutyl)-9-oxa-3,7-diazabicyclo-[3.3.1]non-3-yl ]propyl}amino)benzonitrile, benzenesulphonic acid salt.

12. A composition as claimed in any one of claims 1 to 8, wherein the active ingredient is tert-butyl 2-{7-[(2S)-3-(4-cyanophenoxy)-2-hydroxypropyl ]-9-oxa-3,7-diazabicyclo[3.3.1]non-3-yl}ethylcarbamate or a pharmaceutically-acceptable salt thereof.

13. A composition as claimed in claim 12, wherein the active ingredient is tert-butyl 2- {7-[(2S)-3-(4-cyanophenoxy)-2-hydroxypropyl]-9-oxa-3,7-diazabicyclo [3.3. 1]non-3-yl}ethylcarbamate.

14. A composition as claimed in claim 12, wherein the active ingredient is tert-butyl 2- {7-[(2S)-3-(4-cyanophenoxy)-2-hydroxypropyl]-9-oxa-3,7-diazabicyclo [3.3.1]non-3-yl}ethylcarbamate, methanesulphonic acid salt.

15. A composition as claimed in any one of claims 8 to 14, in which the hydrophilic gelling component comprises maltodextrin, xanthan, scleroglucan, dextran, starch, an alginate, pullulan, hyaloronic acid, chitin, chitosan, albumin, gelatin, poly-L-lysine, sodium poly(acrylic acid), poly(hydroxyethyl methacrylate), carboxypolymethylene, carbomer, polyvinylpyrrolidone, guar gum, gum arabic, gum karaya, gum ghatti, locust bean gum, tamarind gum, gellan gum, gum tragacanth, agar, pectin, gluten, poly(vinyl alcohol), ethylene vinyl alcohol, poly(ethylene oxide), hydroxymethylcellulose, hydroxyethylcellulose, hydroxypropylcellulose, methylcellulose, ethylcellulose, carboxyethylcellulose, ethylhydroxyethylcellulose, carboxymethylhydroxyethylcellulose, hydroxypropylnethylcellulose, hydroxypropylethylcellulose or sodium carboxymethylcellulose, or a copolymer or simple mixture thereof.

16. A composition as claimed in claim 15, wherein the hydrophilic gelling component comprises xanthan, hydroxypropylcellulose, maltodextrin, scleroglucan, carboxypolymethylene, poly(ethylene oxide), hydroxyethylcellulose or hydroxypropylmethylcellulose, or a copolymer or simple mixture thereof.

17. A composition as claimed in claim 16, wherein the hydrophilic gelling component comprises hydroxypropylmethylcellulose.

18. A composition as claimed in claim 17, wherein a 2% solution of the hydrophilic gelling component in water has a viscosity of between 3 and 150,000 cps.

19. A composition as claimed in claim 18, wherein the viscosity is between 10 and 120,000 cps.

20. A composition as claimed in claim 19, wherein the viscosity is between 30 and 50,000 cps.

21. A composition as claimed in claim 20, wherein the viscosity is between 50 and 15,000 cps.

22. A composition as claimed in any one of claims 17 to 21, wherein the hydrophilic gelling component comprises a mixture of hydroxypropylmethylcellulose polymers with different viscosities.

23. A composition as claimed in any one of claims 17 to 22, wherein the hydrophilic gelling component comprises one or more hydroxypropylmethylcellulose polymers fulfilling the United States Pharmacopeia standard substitution types 2208, 2906, 1828 and/or 2910.

24. A composition as claimed in claim 16, wherein the hydrophilic gelling component comprises xanthan.

25. A composition as claimed in claim 24, wherein a 1% solution of the hydrophilic gelling component in water has a viscosity of between 60 and 2,000 cps.

26. A composition as claimed in claim 25, wherein the viscosity is between 600 and 1,800 cps.

27. A composition as claimed in claim 26, wherein the viscosity is between 1,200 and 1,600 cps.

28. A composition as claimed in any one of the preceding claims wherein the composition further comprises a diluent.

29. A composition as claimed in any one of the preceding claims wherein the composition firther comprises a lubricant.

30. A composition as claimed in claim 29, wherein the lubricant is magnesium stearate or sodium stearyl fumarate.

31. A composition as claimed in any one of the preceding claims wherein the composition further comprises a glidant.

32. A composition as claimed in claim 31, wherein the glidant is a colloidal silica.

33. A composition as claimed in any one of the preceding claims wherein the composition further comprises a binder.

34. A composition as claimed in claim 33, wherein the binder is microcrystalline cellulose.

35. A composition as claimed in any one of claims 28 to 34, wherein the total amount of diluent, lubricant, glidant and/or binder in the composition is up to 85% w/w.

36. A composition as claimed in claim 35 wherein the total amount is in the range 0.5 to 45% w/w.

37. A composition as claimed in any one of claims 7 to 36, wherein the amount of polymer or hydrophilic gelling component (as appropriate) in the system is in the range 5 to 99.5% w/w.

38. A composition as claimed in claim 37, wherein the amount is in the range 30 to 70% w/w.

39. A composition as claimed in claim 38, wherein the amount is in the range 35 to 65% w/w.

40. A composition as claimed in any one of the preceding claims in which the amount of active ingredient in the composition is in the range 0.5 to 80% w/w.

41. A composition as claimed in claim 40, wherein the amount is in the range 3 to 70% w/w.

42. A composition as claimed in claim 41, wherein the amount is in the range 5 to 65% w/w.

43. A process for the preparation of a composition as defined in any one of claims 2 to 42, which comprises bringing the active ingredient into association with the carrier.

44. A process as claimed in claim 43, wherein the process of bringing into association comprises wet or dry granulation, direct compression, or a combination of these processes.

45. A composition as defined in any one of claims 1 to 42 for use as a medicament.

46. A composition as defined in any one of claims 1 to 42 for use in the prophylaxis or the treatment of an arrhythmia.

47. The use of a composition as defined in any of one claims 1 to 42 for the manufacture of a medicament for use in the prophylaxis or the treatment of an arrhythmia.

48. The use as claimed in claim 47, wherein the arrhythmia is an atrial or a ventricular arrhythmia.

49. The use as claimed in claim 47, wherein the arrhythmia is atrial fibrillation.

50. The use as claimed in claim 47, wherein the arrhythmia is atrial flutter.

51. A method of prophylaxis or treatment of an arrhythmia which method comprises administration of a composition as defined in any one of claims 1 to 42 to a mammalian patient suffering from, or susceptible to, such a condition.

52. The method as claimed in claim 51, wherein the arrhythmia is an atrial or a ventricular arrhythmia.

53. The method as claimed in claim 51, wherein the arrhythmia is atrial fibrillation.

54. The method as claimed in claim 51, wherein the arrhythmia is atrial flutter.
